SEQUENCE LISTING (21 sequences)

SEQ ID NO: 1 - DNA, 2064 bp, Zea mays
aagtgctaatcctcgtttcgttttgatcaacgaacaaaccctagaccgttaaaacccttatcaatttcgctgaaacggcaaatggaagaagtgtcgtcggagatggaagtggaggttcaaaatcgtcagctttcggattcttctccggctcaaaacgtgaagaaatttgggctcaagaactctattcagaccaacttcggctctgactacgtcttccagattgtcccaaagtaaaagctttctgttctctcttttcgataaccatactaattagtttactgaaagtgaacccaactctggttttgtgacagaatcgattggacagcaatcgcggtttccttatcaaccaacaccgtcaagctttactcgccggtgactggtcagtactacggcgaatgtaaaggccactccgataccgtcaatcagattgcgttttcctccgactcggcggcgtctcctcacgttttgcactcttgttcctccgatggtaccattagatcttgggacactcgaagtttccaacaggtgagtttttttttttttatataaattctgtcgtaaatgtgattccattttcggaactcattgaatttaagttgatcatgggtgtttcaggtttcgcgtattgatactgggaatgatcaggagattttcagcttctcctatggaggtgctgcagataatcttctggctggtggatgcaaagagcaggttaggcatctcaatcctgtggaatcgtgtttttttttttgtttctctaaatgtttaaaaaggagtacactttgagaaagaaaaaaggctagttttgagcattttctatgtttcagcttgttacaatctgaagcgatggtgcaggttcttttgtgggactggagaaactcaaagcaagttgcttgtttagaggaatcccacatggacgacgtcactcaggtttgttgctctctcattctttctgctcaagcgggaaaggaactagtttgttgttagtttatagatgtcaactctgttcttaattataggtacactttgttcctaacaagcccaacaagcttctttccgcttccgtggatggattgatatgtctttttaacactgaaggtgatatcaatgacgatgatcatctggaatctgtaagttttagatttaaggtatcagccttcttgctgcatcaagatagtagatgagagttctgaaccatttctgccttggtcgctctttaggtcatcaacgtgggaacttcaattggcaaaataggtttcctcggagatggctataaaaagctctggtgtctcactcatatagaaactctaaggtactctttcacgccctcctctccaactgtagtctgttacttgtgctgtttttaatttctttactcctttctagtatttggaattgggaagacggaagctgtgaagtcaacctagagaaagctcgggaactcgcatcagatagttggactcaagataatgtaacaatctcattctttcccttctagtaaaacattaacactaaccagaaacctaatggttacttacagagttgaactttcccattactcttttgcaggttgattattttgttgattgtcattgtccaggaggtgaagatttatgggtgatcggtggaacgtgcgcaggaaccgttggttattttccggtgaactacaaacagcctggatcaatcggtaccgctgaagctattcttggtggaggtcacatagatgtagtaaggagcgttctgcagatgccaggcgagtatggaggagctgcagggttatttggatggactgggggtgaagatgggcgactatgttgctggaagtcggatgaggatgctaccgagattaaccggtcttggacttcaagtgagttggttgttaagccgccaaggaaccgcaagaaaaacagacactctccttactagtaaacagatactactttttgggtcttgtgtcttttataaaagttgagactgaagaggacagtttcctttatgtctcttgttcttgttattattaaaaggcaaagttgtttaaatgaaatattgaagaaagattacatc

SEQ ID NO: 2 - DNA, 1652 bp, Zea mays
tgaaatttcttgtgacacgcctcctcttagcaaagatatctggcaacatttagagacagtgaacagttggatttagaacatagagaacttcagacaaacaggtgtacttactggtacaaaaaagagaaacaaaaaagcttggcatacaagcacactaatcattaatggcatatcaaatgctgaaatgcatgctaagtggtaaaactcttgacaagacacatgtagcatataatgaagtgctatatcattacattagtagctacttagataaaaaatataaaacctattcaacatacaataatatttttcacattggaatgaagaattcatactgtcataaagctgtaagaaaactctgttagctaagttcagcaaactgcataagttcattgtaatgttggtcacaaaggcaatcacaaaaatgacatcaccttattgagttgaacttccatttacttatcttttttttctttyttttggcgaaaaggcatctagccgagttggttagacggtctgagtcctcaagtcccgacttcgactaccgtggaagcggatttcaggctgtggttaaaaaaaccttgtctatcccacgccaaagcacgagtctaaggcccggccccagtcgcggtcattctcacatgggctacggtgccgttgtgtatgggtgggacagaggttcaggagtttctcgacctgtgtgagaaggtcttctttttaatacaatgcccaggggctgtcttacctcccgtaggtcgagtttttcttttgaaccggaagaaagttttgagttgcccagtataaagaaaaagtaggtacaaactacggataaatatactcatgttccatcaaatgggatcaatggccaacccaaattcaagcatgaaaataaaaacagaacttgttttagtagtagaatttacaaagcgaacagaagaattagtcatggcgctgtaacgattttaagcagaagtagggcagtgttccaatatgaatgcataagcagtagcaataaagtgcatacggcaacatatattaatctaaacgtaggacaaatgactaaatgaagtgaacaagaatatagtcattatgttaacaggaacatgaaaaaatcgaacataagagaacaaaacagaaatgttgactaatttccattagcatgaacaatatcataaccattttaggagcacacaatcatgggcacaatgttcagatgttctacacctttagcctgtagggtaatatggatatatgattatctttttcattttgattgtgaaataagtagatgtggtagtcttattaagcaaccatacacacatacaaggaaaattgaaatgctgacaagaattttcaataaacataaagtattagaaaacaacctgcttaaagtttcttgtatcccaagctctgacagttccatcagaagaacaagagcagattacttgtggtgacgatggagctgagaaggagatttcatggattgttccttcgtgtcctttacattcccctaggtactgtccggtttctggagaataaaactttagtgcatttgttgacaaggaaactgcaagcgttgagatctcctggcttgttaccagatgtatccaaggagaagcaaaagagcatcagcatcagtactagaagcacagata

SEQ ID NO: 3 - DNA, 20 bp, Artificial Sequence
  GTS1-F1 Primer
gaggagctgcagggttattt

SEQ ID NO: 4 - DNA, 22 bp, Artificial Sequence
  GTS1-R1 Primer
caagacgggttaatctgggtag

SEQ ID NO: 5 - DNA, 21 bp, Artificial Sequence
  TDNA-LB Primer
ccgtctcactggtgaaaagaa

SEQ ID NO: 6 - DNA, 22 bp, Artificial Sequence
  GTS1-F2 Primer
ctgaaacggcaaatggaagaag

SEQ ID NO: 7 - DNA, 21 bp, Artificial Sequence
  GTS1-R2 Primer
ctatgttgctggaagtcggat

SEQ ID NO: 8 - DNA, 34 bp, Artificial Sequence
  Act2-F Primer
gcggatccatggctgaggctgatgatattcaacc

SEQ ID NO: 9 - DNA, 36 bp, Artificial Sequence
  Act2-R Primer
cgtctagaccatggaacattttctgtgaacgattcc

SEQ ID NO: 10 - DNA, 22 bp, Artificial Sequence
  Primer
tccatcgtcaccacaagtaatc

SEQ ID NO: 11 - DNA, 21 bp, Artificial Sequence
  Primer
cggtcatcaggcacagaatag

SEQ ID NO: 12 - DNA, 49 bp, Artificial Sequence
  Primer
gcggccgctaatacgactcactataggtccatcgtcaccacaagtaatc

SEQ ID NO: 13 - DNA, 48 bp, Artificial Sequence
  Primer
gcggccgctaatacgactcactataggcggtcatcaggcacagaatag

SEQ ID NO: 14 - DNA, 22 bp, Artificial Sequence
  Actin-F Primer
gaggatgatgatgcagcgatag

SEQ ID NO: 15 - DNA, 18 bp, Artificial Sequence
  Actin-R Primer
tttggcgcggaatgaaga

SEQ ID NO: 16 - protein, 392 aa, Arabidopsis thaliana
MEEVSSEMEVEVQNRQLSDSSPAQNVKKFGLKNSIQTNFGSDYVFQIVPKIDWTAIAVSLSTNTVKLYSPVTGQYYGECKGHSDTVNQIAFSSDSAASPHVLHSCSSDGTIRSWDTRSFQQVSRIDTGNDQEIFSFSYGGAADNLLAGGCKEQVLLWDWRNSKQVACLEESHMDDVTQVHFVPNKPNKLLSASVDGLICLFNTEGDINDDDHLESVINVGTSIGKIGFLGDGYKKLWCLTHIETLSIWNWEDGSCEVNLEKARELASDSWTQDNVDYFVDCHCPGGEDLWVIGGTCAGTVGYFPVNYKQPGSIGTAEAILGGGHIDVVRSVLQMPGEYGGAAGLFGWTGGEDGRLCCWKSDEDATEINRSWTSSELVVKPPRNRKKNRHSPY

SEQ ID NO: 17 - protein, 351 aa, Oryza sativa
MPSASYFIYLVTSQENSTLAVSLSTNALKLYSPATGQFLGECTGHSGSIHEISFSAPSSPQVICSCSSDGTIRAWDTRSFKQISLLRGSQELFSFSFGGSSGNLLAAGSNSQVLLWDWRNSKQLACLEESHMDDVTQVKFAPHQHSKLISAAVDGLICVFDTDGDMNEDNHLLSVMNVETSVAKVGFFGNTYQKIWCLSHIETLSTWDWNDGSRELNIDDARSLATDRWNLDHVDYFVDCHYSLPDDKLWVIGGTTAGTIGYFPVRAGLEGAIGSAEAILEGGHTDVVRTVYPAANTHTGQNRGIFGWTGGEDGRLCCWRSGEIAEMNKSWISSSLVSRVQKKAKCRQQPY

SEQ ID NO: 18 - protein, 356 aa, Zea mays
MGPKIIHLFPHLIYREISTLAVSLSTNALKFYSPETGQYLGECKGHEGTIHEISFSAPSSPQVICSCSSDGTVRAWDTRNFKQISLLRGGVSQEMFTFAFGGSNGSLLAAGSNAQVLFWDWRSSRQIACLEESHMDDVTQVKFTPDQQSKLISAAVDGLICVFDTDGDIDDDNHLLSVMNAETSVAKVGFFGNKYKKLWCLTHIETLSVWDWNDGARELNIEDARSLATDRWNLDHVDYFVDCHYSVPDDRLWLIGGTTAGTLGYFPVRNDHDGPIGSAEAILEGGHTGVVRSVYPSSGIHGRLGQNRGIFGWTGGEDGRLCCWRSDEIAETNKSWISSSLVWKQQKKTKNRHQPY

SEQ ID NO: 19 - protein, 387 aa, Homo sapiens
MEKIEEQFANLHIVKCSLGTKEPTYLLGIDTSKTVQAGKENLVAVLCSNGSIRIYDKERLNVLREFSGYPGLLNGVRFANSCDSVYSACTDGTVKCWDARVAREKPVQLFKGYPSNIFISFDINCNDHIICAGTEKVDDDALLVFWDARMNSQNLSTTKDSLGAYSETHSDDVTQVRFHPSNPNMVVSGSSDGLVNVFDINIDNEEDALVTTCNSISSVSCIGWSGKGYKQIYCMTHDEGFYWWDLNHLDTDEPVTRLNIQDVREVVNMKEDALDYLIGGLYHEKTDTLHVIGGTNKGRIHLMNCSMSGLTHVTSLQGGHAATVRSFCWNVQDDSLLTGGEDAQLLLWKPGAIEKTFTKKESMKIASSVHQRVRVHSNDSYKRRKKQ

SEQ ID NO: 20 - protein, 386 aa, Ratus norvegicus
MEKIKDQFANLHILRRSSEPKEPTYLLGIDTSKTVPAEKGGLVAVLCSNGSIRIYDKETLNLLREFSGSPGLLNGVRFANSCDNVYSASTDGTVKCWDARLASEKPAQLFKGYPSNIFISFDVNCKDHIICAGAEKVEDDALLVFWDARFTSQDLSTRDPLGAYSETHSDDITQVRFHPSNPNMVVSGSTDGLVNVFDLSVDNEEDALVATCNSVSSVSCIGWCGRDYKQIYCMTHDEGFCWWDLNHLDTDEPITCLNIQDVRDVTDVKEGHLDYLIGGLYHENMDRLFVIGGTNLGKIHLLSCTKTGLSHVTSLQGGHAATVRSFCWTVSEDSLLTGGEDAQLLLWKPGAVEKTFTKKDSLKIASSVQQRVRVHSSDSYKKRKQQ

SEQ ID NO: 21 - protein, 386 aa, Mus musculus
MEKIEDQFASLHIVRRSSEPKEPTYLLGIDTSKTVQADKGGLVAVLCSNGSIRIYDKETLHLLREFGGSPGLLSGVSFANSCDSVYSASTDGTVKCWDARGASEKPVQLFKGYPSCSFISFDVNCKDHVICAGAEKVDEDALLVFWDARFTSQDLSTRDPLGAYSETHSDDITQVRFHPSNPNLVVSGSTDGLVNVFDLSADKEEDALVATCNSVSSVSCIGWCGKDYKQIYCMTHDEGFCWWDLNHLDTDEPITCLNIQDVREITDVKDGHLDYLIGGLYHEKMDRLFVIGGTNTGKIHLLSCTSAGLTHVTSLHGGHAATVRSFCWNVSEDSLLTGGEDAQLLLWKPGAMEKTFTKKDSLKIASSVQQRVRVHSSDSYKKRKQQ

(19) United States
(12) Patent Application Publication
(10) Pub. No.: US 2017/0211085 A1
(43) Pub. Date: Jul. 27, 2017

(54) GIGANTUS1 (GTS1) GENE AND METHODS OF USE THEREOF FOR IMPROVING BIOMASS ACCUMULATION AND HIGHER YIELD IN PLANTS

(71) Applicant: Rutgers, The State University of New Jersey, New Brunswick, NJ (US)

(72) Inventors: Simeon Kotchoni, Marlton, NJ (US); William M. Saidel, Cherry Hill, NJ (US); Benedetto Piccoli, Cherry Hill, NJ (US); Emma W. Gachomo, Marlton, NJ (US)

(21) Appl. No.: 15/008,026

(22) Filed: Jan. 27, 2016

(51) Int. Cl. C12N 15/82; G01N 33/50; C07K 14/415

(52) U.S. Cl. CPC C12N 15/8261; C12N 15/8218; C07K 14/415; G01N 33/5097

(57) ABSTRACT

Compositions and methods are provided for enhancing growth, seed germination, and biomass production in transgenic plants expressing reduced levels of GIGANTUS1 (gts1).

This application claims priority to U.S. Provisional Application No. 62/108,481 filed Jan. 27, 2015, the entire contents being incorporated herein by reference as though set forth in full.

### FIELD OF THE INVENTION

This invention relates to the fields of molecular biology and the generation of transgenic plants with improved phenotypes. More specifically, the invention provides expression vectors encoding GTS1 and homologs thereof for the creation of transgenic plants which exhibit increased biomass.

### BACKGROUND OF THE INVENTION

Numerous publications and patent documents, including both published applications and issued patents, are cited throughout the specification in order to describe the state of the art to which this invention pertains. Each of these citations is incorporated herein by reference as though set forth in full.

Transducin/WD40 repeat proteins are prominent features within proteins that mediate diverse protein-protein interactions, including those involved in scaffolding and the cooperative assembly and regulation of dynamic multi-subunit complexes [1, 2]. The common and defined feature of these proteins are short ˜40 amino acid motifs, typically ending by Trp-Asp sequence, and usually composed of a 7-8 bladed beta-propeller fold. However, a diversity of proteins has been found with 4 to 16 repeated units [3]. The low level of sequence conservation and functional diversity of WD40 domains make their identification difficult.

Repeated WD40 domains play central roles in biological processes such as cell division and cytokinesis, apoptosis, light signaling and vision, cell motility, flowering, floral development, meristem organization, protein trafficking, cytoskeleton dynamics, chemotaxis, nuclear export to RNA processing, chromatin modification, and transcriptional mechanism [4]. They act as a site for protein-protein interaction, where the specificity of the proteins is determined by the sequences outside the repeats themselves. Their functional importance resides largely on the protein surfaces. They serve as multi-interacting platforms in cellular networks for the assembly of protein complexes or mediators of transient interplay among other proteins [5]. Structural studies suggest that this property stems from their ability to interact with diverse proteins, peptides or nucleic acids using multiple surfaces, where the most common peptide interaction site of WD40 proteins is located on the top surface of the propeller close to the central channel [6].

Although WD40 proteins also are present in bacteria, e.g. Thermomonospora curvata [7] and Cyanobacterium synechocystis [8], WD40 domains are among the ten most abundant domain types across eukaryotic proteomes, and interactome studies suggest that they are among the most promiscuous interactors. Despite several WD40-containing proteins acting as key regulators of plant-specific developmental events, WD40 domains have been given less research attention compared to other common domains, for example kinase domains. Up to now, no comprehensive 3D structural analyses of WD40 domain containing proteins have been performed. Thus key interacting partners and the relevance of different WD40 domains which mediate different biological functions have yet to be elucidated[9]. Furthermore, in contrast to other members of the β-propeller family, and despite being crucial for and residing in enzymatic complexes, no WD40 protein has been reported to possess catalytic activity.

### SUMMARY OF THE INVENTION

In accordance with the present invention, a novel nucleic acid molecule, GTS1, is provided. GTS1 encodes the GIGANTUS1 protein (GTS1) which is a member of the transducing/WD40 protein superfamily. Surprisingly, the present inventors have discovered that mutation or disruption of the nucleic acid encoding this protein in transgenic plants produces plants exhibiting several desirable phenotypes, including, without limitation, increased biomass, early flowering time, increased seed production and enhanced growth development.

In one aspect of the invention, an isolated nucleic acid molecule comprising a GTS1-encoding nucleic acid sequence or a homolog thereof having at least 90% identity is provided. In a preferred embodiment, the nucleic acid molecule is isolated from a plant and has the GTS1-encoding nucleic acid sequence of SEQ ID NO: 1 or SEQ ID NO: 2 or a sequence having at least 70-75, 80-85, 90, 91, 92, 93, 95, 98 or 99% identity therewith. Optionally, and in certain cases preferably, the nucleic acids described above comprise a mutation or disruption (e.g., introduction of a stop codon or deletion of one, two, three or five or more nucleotides) that alters the function of the encoded protein. Preferably, the nucleic acid molecules of the invention may be inserted into an expression vector, such as a plasmid or viral vector, and may further be transformed into a host cell or plants. Exemplary host cells and plants include Arabidopsis, rice, maize, wheat, tomato, potato, barley, canola, bacteria, yeast, insect and mammalian cells.

According to yet another aspect of the invention, methods for identifying agents which modulate GTS1 activity and expression are provided. The methods comprise introducing GTS1-encoding nucleic acid molecules into a host cell, treating the cells or resulting plant with agents suspected of modulating GTS1 activity and expression and assaying GTS1 activity and expression levels in the presence and absence of the agent.

According to a preferred aspect of the invention, a method is provided to enhance biomass production in a plant comprising reducing expression levels of GTS1-encoding nucleic acid molecules having the sequence of SEQ ID NO: 1 or SEQ ID NO: 2 and homologs thereof. In another embodiment of the invention, GTS1 expression is reduced by the addition of an agent that inhibits GTS1 expression in a host cell.

According to yet another aspect of the invention, a method is provided to inhibit GTS1 function in a plant. This method comprises introducing a mutated GTS1-encoding nucleic acid molecule into a plant which encodes a non-functional GTS1 protein. In one embodiment, the GTS1-encoding nucleic acid molecule is an antisense molecule of SEQ ID NO: 1 or SEQ ID NO: 2. Alternatively, methods for inhibition of GTS1 expression comprise introduction of nucleic acid constructs effective to cause dsRNA mediated gene silencing of the GTS1 gene.

According to another aspect of the present invention, transgenic plants comprising an altered SEQ ID NO: 1 or SEQ ID NO: 2 are provided. The transgenic plants of the invention exhibit increased biomass, early flowering times increased seed production and robust early growth. Knock-out plants wherein GTS1 expression has been eliminated are also provided. Also within the scope of the invention are transgenic plants comprising variants of SEQ ID NO: 1 or SEQ ID NO: 2 encoding proteins having altered GTS1 function, e.g., sequences including a stop codon which prematurely truncates the protein or sequences containing between 1, 2, 3, 4, 5, or 10 to 15 amino acid deletions.

In a further embodiment of the invention, the screening methods provided above are performed employing nucleic acids, proteins and antibodies encoded by the GTS1 homologs and functional orthologs identified in maize and rice and other species. These functionally equivalent homologs to GTS1 may also be used to modulate biomass production in higher plants.

In a final aspect of the invention, improved maize germplasm is provided wherein the gts 1 gene has been knocked out using the RNAi GTS1construct shown in FIG. 19. Generation of transgenic maize callus into plants exhibiting improved growth rates, biomass accumulation, flowering time, crop yield and resistance to drought stress is also within the scope of the present invention.

The patent or application file contains at least one drawing executed in color. Copies of this patent or patent application publication with color drawing(s) will be provided by the Office upon request and payment of the necessary fee.

### BRIEF DESCRIPTION OF THE DRAWINGS

FIGS. 1A-1C: Tissue specific expression profile and Phylogenetic analysis of GTS1. FIG. 1A. GENEVESTIGATOR-Microarray data showing highly expressed GTS1 gene in embryo, root apical meristem, root tip, abscission zone and shoot apex [24]. FIG. 1B. Experimental expression analysis of GTS1 showing increased transcript accumulation in germinated seed, young and developed rosette leaves and developed flower. FIG. 1C. Phylogenetic relationships between plant genes containing WD40 repeat domains. The GTS1 genes of Arabidopsis, rice, and maize (shown in red), belong to a subclade of the dominate clade (gray branches) containing most of the plant genes listed. Genbank accession numbers used for all genes with the exception of Arabidopsis and rice which use the Gene ID number from the SALK database.

FIG. 2: Hierarchical clustering microarray expression analysis of GTS1 and other selected tissue specific genes. Depicted squares display genes with similar tissue specific expression pattern with GTS1 (see Table 2 for detail description). Data analysis was retrieved from Genevestigator [24].

FIG. 3: The plant GTS1 proteins are most similar to the animal Wdr89 protein. ClustalW alignment of the plant GTS1 proteins from Arabidopsis (AtGTS1; SEQ ID NO: 16), Oryza (OsGTS1; SEQ ID NO: 17), and Zea (ZmGTS1; SEQ ID NO: 18), with the animal Wdr89 proteins from Homo sapiens (HsWdr89; (SEQ ID NO: 19), Ratus norvegicus (RnWdr89; SEQ ID NO: 20), and Mus musculus (MmWdr89; SEQ ID NO: 21) show several conserved residues across the entire length of the protein (grey shaded residues). Numbers in parenthesis are the percent identity that the corresponding protein shares with AtGTS1. WD40 repeat domains are underlined for all three plant GTS1 proteins. The section in red box delimits the conserved fragment (WD40 domain) used to construct the GTS1-RNAi vector for knockout gene in developing the gts1 maize germplasm in this work.

FIGS. 4A-4H: Physical map of GTS1 knockout gene and phenotypic characterization of gts1 mutant. FIG. 4A. The GTS1 gene with the positions of exons (numbered black rectangles) and introns (thick lines) are represented. The 5′ and 3′ untranslated regions are depicted in white rectangles. The location of the gts1 T-DNA insertion is shown using an inverted black triangle. The names and locations of primers used for RT-PCR experiments are also indicated. Bar=0. 5 kb. FIG. 4B. The T-DNA insertion causes a knockout expression of the gene. The quality of the

RNA and the loading control was assayed by monitoring ACTIN gene expression. FIGS. 4C-FIG. 4F GTS1 negatively controls seed germination. gts1 mutant germinated faster at 1 and 3 days after incubation in water (FIG. 4D, FIG. 4F) than the wild type (FIG. 4C, FIG. 4E). FIGS. 4G-4H. GTS1 controls biomass accumulation and growth development in Arabidopsis. FIG. 4H. Growth rate of gts1 is faster than that of WT (FIG. 4G) at 15 DAG. gts1 shows larger leaf area (biomass) (FIG. 4H) than WT (FIG. 4G). DAG=Days after germination.

FIGS. 5A-5B: Mutation in GTS1 gene promotes early flowering, growth development and biomass accumulation. FIG. 5A. a faster growth of gts1 mutant compared to WT is depicted with gts1 displaying a taller phenotype than WT. FIG. 5B. gts1 mutant flowers earlier than WT as depicted by a reduced number of gts1 rosette leaves compared to WT at bolting time. Gts1 mutant accumulates higher cell biomass than WT as shown by a bigger overall gts1 rosette leaf area compared to WT (FIG. 5B).

FIG. 6: Correlation of expression pattern between GTS1 and the ribosomal protein. Samples whose contribution is more than 1.0 are outputted. The probe pair giving highest correlation is selected from all combination between the probes for the two loci, 266466_at (GTS1) and 258410_at (L19e) respectively. Sample contribution score is calculated as a product of z-scored expression values. Average of the score is pearsons correlation coefficient.

FIGS. 7A-7B: Structure of GTS1, a WD40 repeat protein. FIG. 7A. Top, bottom and side views of the seven-bladed β-propeller structure (most stable form) made by using PyMol software on the world wide web at www. Pymol.org. with the N-terminal and C-terminal regions in blue and red color, respectively. A depicted model is included to show the basic WD40 β-sheet structures conformation of the cylinder structure with a tunnel-like structure in the centre that communicate both top and bottom sides. FIG. 7B. 180° rotated views of the electrostatic potential representation on the GTS1 protein surface. The surface colors are clamped at red (−10) or blue (+10).

FIGS. 8A-8B: Conservational and ligand binding domain analysis of GTS1, a WD40 repeat protein. FIG. 8A. Consurf-conservational analysis of GTS1 protein showed in two individual views rotated 180°. The conserved and variable residues are presented as space-filled models and colored according to the conservation scores. The interacting area of the protein with ribosomal counterparts has been highlighted by a red discontinue line. FIG. 8B. Detailed view of the ligand-binding area of GTS1 with a peptide, and the spatial distribution of the interacting residues.

FIGS. 9A-9D: Conservational and ligand binding domain analysis of ribosomal Nop16 and L19e proteins. FIG. 9A. Consurf-conservational analysis of Nop16 protein showed in two individual views rotated 45°. The conserved and variable residues are presented as space-filled models and colored according to the conservation scores. The interacting areas of the protein with GTS1 protein and ribonucleic acid have been highlighted in red and blue color, respectively. FIG. 9B. Detailed views of the ligand-binding area of Nop16 protein with a chain of ribonucleic acid, and the spatial distribution of the interacting residues. FIG. 9C. Consurf-conservational analysis of L19e protein showed in two individual views rotated 45°. The conserved and variable residues are presented as space-filled models and colored according to the conservation scores. The interacting areas of the protein with GTS1 protein and ribonucleic acid have been highlighted in red and blue color, respectively. FIG. 9D. Detailed views of the ligand-binding area of L19e protein with ribonucleic acid and the spatial distribution of the interacting residues depicted in both detailed views.

FIGS. 10A-10C: Analysis of the interaction between GTS1, a WD40 repeat and Nop16 proteins. FIG. 10A. The complex between GTS1 (blue surface and cartoon representation) and Nop16 (white/gray surface and cartoon representation) from the top view. FIG. 10B. Surface/cartoon structure rotated 90° in blue (GTS1) and white/gray (Nop16) are depicted, and highlight the large interacting surface between both proteins. FIG. 10C. Electrostatic potential depicted in both interacting partners, where has been highlighted both areas involved in the interaction by light-blue discontinue arrows. The surface colors are clamped at red (−10) or blue (+10).

FIGS. 11A-11C.:Analysis of the interaction between GTS1, a WD40 repeat and L19e proteins. FIG. 11A. The complex between GTS1 (green surface and cartoon representation) and L19e (white/gray cartoon representation) from a lateral view. FIG. 11B. Surface/cartoon structure rotated 45° in green (GTS1) and white/gray (L19e) are depicted, and highlight the two interacting surfaces between both proteins. FIG. 11C. Electrostatic potential represented in both interacting partners, where has been highlighted both areas involved in the interaction by black discontinue arrows. A detailed view of this interaction has been depicted. The surface colors are clamped at red (−10) or blue (+10).

FIG. 12: gts1 mutant produces more seeds than WT. Number of siliques (seed pods) produced at indicated days after germination. Comparative production of siliques between WT and gts1 is depicted. No silique was produced by WT at 31 days after germination Means ±STDEV of plants (n=6) per genotype are shown. Significant differences in comparison analysis are indicated with asterisks: * P<0.05.

FIGS. 13A-13H: Cell shape patterning/characterization to follow the effect of gene knockout on growth of WT vs. mutant plants. FIG. 13A. Cells of the WT and gts1 mutants were treated with FM464 and images were obtained as a set of confocal images (a stack). A stack of images was opened and collapsed to generate a single image of a field. FIG. 13B. Cells with complete boundaries within the single image were identified and numbered. FIG. 13C. Each complete cell was identified in the image and the image around the cell was erased. A fluorescent boundary of the isolated cell was thresholded to provide a single cell boundary. FIG. 13D. All extraneous pixels and non-essential dead-end projections were removed to generate the single cell. FIG. 13E. Overlapping FIGS. 13A and 13D. FIG. 13F. A single pixel wide representation of the cell boundary was obtained by applying a Canny Edge Detector. FIG. 13G. The single pixel boundary in FIG. 13F is now a boundary equal to the width of the cell. FIG. 13H. Overlapping FIGS. 13G and 13A.

FIG. 14: Fractal dimension vs. cell perimeter (left panel) and cell area (right panel) to follow the growth patterning of plants at a cellular level.

FIG. 15: Area vs. Fractal dimension of WT vs. gts1 cells.

FIGS. 16A-16B.: WT (FIG. 16A) vs. gts1 (FIG. 16B) cell shape phenotype. WT cells are bigger with more complex circularity than gts1 cells. (n=50 cells analysed).

FIGS. 17A-17C: Quantitative proteome profiling of WT vs. gts1 mutant. FIG. 17A-FIG. 17B. Different peptide ID signature from WT and GTS1 responsible for GTS1 phenotypic traits. Arrows (1, 2 & 3) depict selected quantitative peptides differentially accumulated in both WT and gts1 mutant, while arrow heads depict peptides solely accumulated in one genotype and absent in the other. FIG. 17C. Heatmap profile representation of quantitative peptide peak ID generated form A & B panels. In this LC-MS/MS experiments, cluster I represents quantitatively down regulated proteins in WT vs. gts1, while cluster II depicts overexpressed proteins in WT vs. gts1. Cluster III represents differentially expressed proteins in both WT and gts1 mutant. The data revealed a plethora of proteins controlling polarized cell growth and expansion, defense response and abiotic stress tolerance in plants.

FIGS. 18A-18D:Callus induction from immature B73 maize seedling. FIG. 18A. 5 DAG agar grown maize seedling used for the callus induction is depicted with ˜1 cm of bud length germination. FIG. 18B. Callus production at three days post induction, FIG. 18C. Callus production at five days post induction, FIG. 18D. Callus production at three days post induction at seven days post induction.

FIG. 19: Genetic map of the RNAi GTS1 gene knockdown construct generated by transforming wild type (B73) maize callus with recombinant MWpBacFPNS plasmid expressing a double strand GTS1 cDNA operably linked to a T7 promoter control at both 5′ and 3′ ends.

FIG. 20: Generation of transgenic maize callus with GTS1-RNAi construct. Lane 1=Wild type, lane 2=transformed callus. The presence of amplified band in lane 2 confirm the successful insertion of the construct into the genomic DNA. Lower panel=Actin control.

FIG. 21: Quantitative gene expression analysis of WT and RNAi knockout mutant calli using Real Time Q-PCR. Arrow heads indicate the two selected mutants (Zm-gts1-4 and Zm_gts1-8) with the highest GTS1 suppression pattern compared to WT.

### DETAILED DESCRIPTION OF THE INVENTION

WD40 domains have been found in a plethora of eukaryotic proteins, acting as scaffolding molecules assisting proper activity of other proteins, and participating in a variety of different multi-cellular processes. They comprise several stretches of 44-60 amino acid residues often terminating with a WD di-peptide. They act as a site of protein-protein interactions or multi-interacting platforms, driving the assembly of protein complexes or as mediators of transient interplay among other proteins. In Arabidopsis, members of WD40 protein superfamily are known as key regulators of plant-specific events, biologically playing important roles in development and also during stress signaling.

Using reverse genetic and protein modeling approaches, we characterize GIGANTUS1 (GTS1), a new member of WD40 repeat protein in Arabidopsis thaliana and provide evidence of its role in controlling plant growth development. GTS1 is highly expressed during embryo development and negatively regulates seed germination, biomass yield and growth improvement in plants. Structural modeling analysis suggests that GTS1 folds into a β-propeller with seven pseudo-symmetrically arranged blades around a central axis. Molecular docking analysis shows that GTS1 physically interacts with two ribosomal protein partners, a component of ribosome Nop16, and a ribosome-biogenesis factor L19e through β-propeller blade 4 to regulate cell growth development. Clearly, GIGANTUS1 provides a promising target for engineering transgenic plants having higher biomass and improved growth development for plant-based bioenergy production.

The definitions set forth below are provided to facilitate an understanding of the present invention.

As used herein, a “GIGANTUS 1 (gts1) encoding nucleic acid” refers a nucleic acid encoding a member of the WD40 protein super family which modulates biomass accumulation, early flowering increased seed production and robust early growth in plants. Surprisingly, gene knockout of gts1 leads to more than a three-fold increase in biomass accumulation and a five-fold increase in seed production. SEQ ID NOS: 1 and 2 provided herein encode gts1.

“Transgenic plant” refers to a plant whose genome has been altered by the introduction of at least one heterologous nucleic acid molecule.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production. The terms “percent similarity”, “percent identity” and “percent homology” when referring to a particular sequence are used as set forth in the University of Wisconsin GCG software program.

The term “substantially pure” refers to a preparation comprising at least 50 60% by weight of a given material (e.g., nucleic acid, oligonucleotide, protein, etc.). More preferably, the preparation comprises at least 75% by weight, and most preferably 90 95% by weight of the given compound. Purity is measured by methods appropriate for the given compound (e.g. chromatographic methods, agarose or polyacrylamide gel electrophoresis, HPLC analysis, and the like).

A “replicon” is any genetic element, for example, a plasmid, cosmid, bacmid, plastid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element. An “expression vector” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide,” as used herein refers to sequences, primers and probes of the present invention, and is defined as a nucleic acid molecule comprised of two or more ribo or deoxyribonucleotides, preferably more than three. The exact size of the oligonucleotide will depend on various factors and on the particular application and use of the oligonucleotide.

The phrase “specifically hybridize” refers to the association between two single stranded nucleic acid molecules of sufficiently complementary sequence to permit such hybridization under pre determined conditions generally used in the art (sometimes termed “substantially complementary”). In particular, the term refers to hybridization of an oligonucleotide with a substantially complementary sequence contained within a single stranded DNA or RNA molecule of the invention, to the substantial exclusion of hybridization of the oligonucleotide with single stranded nucleic acids of non complementary sequence.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single stranded or double stranded. The exact length of the probe will depend upon many factors, including temperature, source of probe and method of use. For example, for diagnostic applications, depending on the complexity of the target sequence, the oligonucleotide probe typically contains 15 25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non complementary nucleotide fragment may be attached to the 5′ or 3′ end of the probe, with the remainder of the probe sequence being complementary to the target strand. Alternatively, non complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “promoter region” refers to the 5′ regulatory regions of a gene (e.g., CaMV 35S promoters and/or tetracycline inducible repressor/operator gene promoters).

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radioimmunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell or plant.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “DNA construct” refers to a genetic sequence used to transform plants and generate progeny transgenic plants. These constructs may be administered to plants in a viral or plasmid vector. Other methods of delivery such as Agrobacterium T-DNA mediated transformation and transformation using the biolistic process are also contemplated to be within the scope of the present invention. The transforming DNA may be prepared according to standard protocols such as those set forth in “Current Protocols in Molecular Biology”, eds. Frederick M. Ausubel et al., John Wiley & Sons, 1995.

The phrase “double-stranded RNA mediated gene silencing” refers to a process whereby target gene expression is suppressed in a plant cell via the introduction of nucleic acid constructs encoding molecules which form double-stranded RNA structures with target gene encoding mRNA which are then degraded.

The term “co-suppression” refers to a process whereby expression of a gene, which has been transformed into a cell or plant (transgene), causes silencing of the expression of endogenous genes that share sequence identity with the transgene. Silencing of the transgene also occurs.

A low molecular weight “peptide analog” shall mean a natural or mutant (mutated) analog of a protein, comprising a linear or discontinuous series of fragments of that protein and which may have one or more amino acids replaced with other amino acids and which has altered, enhanced or diminished biological activity when compared with the parent or nonmutated protein.

The present invention also includes active portions, fragments, derivatives and functional or non-functional mimetics of GTS1-related polypeptides, or proteins of the invention. An “active portion” of such a polypeptide means a peptide that is less than the full length polypeptide, but which retains measurable biological activity.

A “fragment” or “portion” of an GTS1-related polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids. Fragments of the GTS1-related polypeptide sequence, antigenic determinants, or epitopes are useful for eliciting immune responses to a portion of the GTS1-related protein amino acid sequence for the effective production of immunospecific anti-GTS1 antibodies.

Different “variants” of the GTS1-related polypeptides exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include but are not limited to: (a) variants in which one or more amino acids residues are substituted with conservative or non-conservative amino acids, (b) variants in which one or more amino acids are added to the polypeptide, (c) variants in which one or more amino acids include a substituent group, and (d) variants in which the polypeptide is fused with another peptide or polypeptide such as a fusion partner, a protein tag or other chemical moiety, that may confer useful properties to the GTS1-related polypeptide, such as, for example, an epitope for an antibody, a polyhistidine sequence, a biotin moiety and the like. Other GTS1-related polypeptides of the invention include variants in which amino acid residues from one species are substituted for the corresponding residue in another species, either at the conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques are known to the person having ordinary skill in the art. To the extent such allelic variations, analogues, fragments, derivatives, mutants, and modifications, including alternative nucleic acid processing forms and alternative post-translational modification forms, result in derivatives of the GTS1-related polypeptide that retain any of the biological properties of the GTS1-related polypeptide, they are included within the scope of this invention.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by the trained artisan, and are contemplated to be within the scope of this definition.

A “clone” or “clonal cell population” is a population of cells derived from a single cell or common ancestor by mitosis.

A “cell line” is a clone of a primary cell or cell population that is capable of stable growth in vitro for many generations.

The following description sets forth the general procedures involved in practicing the present invention. To the extent that specific materials are mentioned, it is merely for purposes of illustration and is not intended to limit the invention. Unless otherwise specified, general biochemical and molecular biological procedures, such as those set forth in Sambrook et al., Molecular Cloning, Cold Spring Harbor Laboratory (1989) (hereinafter “Sambrook et al.”) or Ausubel et al. (eds) Current Protocols in Molecular Biology, John Wiley & Sons (1997) (hereinafter Ausubel et al.) are used.

### Preparation of GTS1 Encoding Nucleic Acid Molecules:

Nucleic acid molecules of the invention encoding GTS1 polypeptides may be prepared by two general methods: (1) synthesis from appropriate nucleotide triphosphates, or (2) isolation from biological sources. Both methods utilize protocols well known in the art. The availability of nucleotide sequence information, such as the DNA sequences encoding GTS1, enables preparation of an isolated nucleic acid molecule of the invention by oligonucleotide synthesis. Synthetic oligonucleotides may be prepared by the phosphoramidite method employed in the Applied Biosystems 38A DNA Synthesizer or similar devices. The resultant construct may be used directly or purified according to methods known in the art, such as high performance liquid chromatography (HPLC). Specific probes for identifying such sequences as the GTS1 encoding sequence may be between 15 and 40 nucleotides in length. For probes longer than those described above, the additional contiguous nucleotides are provided within SEQ ID NO: 1 or SEQ ID NO: 2. Preferably, such probes are detectably labeled to facilitate identification of hybridizing sequences.

Additionally, cDNA or genomic clones having homology with GTS1 may be isolated from other species using oligonucleotide probes corresponding to predetermined sequences within the GTS1 nucleic acids of the invention. Such homologous sequences encoding GTS1 may be identified by using hybridization and washing conditions of appropriate stringency as described in Example I below. For example, hybridizations may be performed, according to the method of Sambrook et al., Molecular Cloning, Cold Spring Harbor Laboratory (1989), using a hybridization solution comprising: 5×SSC, 5× Denhardt's reagent, 1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology (Sambrook et al., 1989) is as follows:

Tₘ=81.5° C.+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The nucleic acid molecules described herein include cDNA, genomic DNA, RNA, and fragments thereof which may be single- or double-stranded. Thus, nucleic acids are provided having sequences capable of hybridizing with at least one sequence of a nucleic acid sequence, such as selected segments of the sequences encoding GTS1. Also contemplated in the scope of the present invention are methods of use for oligonucleotide probes which specifically hybridize with the DNA from the sequences encoding GTS1 under high stringency conditions. Primers capable of specifically amplifying the sequences encoding GTS1 are also provided. As mentioned previously, such oligonucleotides are useful as primers for detecting, isolating and amplifying sequences encoding GTS1.

Antisense nucleic acid molecules which may be targeted to translation initiation sites and/or splice sites to inhibit the expression of the GTS1 gene or production of its encoded protein are also provided. Such antisense molecules are typically between 15 and 30 nucleotides in length and often span the translational start site of GTS1 mRNA molecules. Antisense constructs may also be generated which contain the entire GTSI cDNA in reverse orientation. Alternatively, the GTS1 gene may be silenced using a construct that contains both sense and complementary antisense sequences separated by an intron sequence. This “intron-spliced hairpin RNA” approach results in total silencing of the targeted gene by forming double stranded RNA (dsRNA) structures which are degraded by the host cell machinery (Smith et al., 2000; Wesley et al., 2001).

Also provided in accordance with the present invention are transgenic plants containing the aforementioned GTS1-encoding nucleic acids, or fragments or derivatives thereof. Such transgenic plants and their utility in increasing biomass accumulation are described in greater detail below.

The present invention also provides antibodies capable of immunospecifically binding to proteins of the invention. Polyclonal or monoclonal antibodies directed toward GTS1 may be prepared according to standard methods. Monoclonal antibodies may be prepared according to general methods of Köhler and Milstein, following standard protocols. In a preferred embodiment, antibodies are prepared, which react immunospecifically with various epitopes of GTS1.

Polyclonal or monoclonal antibodies that immuno-specifically interact with GTS1 may be utilized for identifying and purifying such proteins. For example, antibodies may be utilized for affinity separation of proteins with which they immunospecifically interact. Antibodies may also be used to immunoprecipitate proteins from a sample containing a mixture of proteins and other biological molecules.

### Uses of GTS1 Nucleic Acid Molecules and Proteins

### A. Nucleic Acids Encoding GTS1-Related Proteins

Nucleic acids encoding GTS1 proteins may be used for a variety of purposes in accordance with the present invention. DNA, RNA, or fragments thereof encoding GTS1 proteins may be used as probes to detect the presence of and/or expression of such genes. Methods in which nucleic acids encoding GTS1 proteins may be utilized as probes for such assays include, but are not limited to: (1) in situ hybridization; (2) Southern hybridization (3) Northern hybridization; and (4) assorted amplification reactions such as polymerase chain reactions (PCR).

The nucleic acids of the invention may also be utilized as probes to identify related genes from other plant species, animals and microbes. As is well known in the art, hybridization stringencies may be adjusted to allow hybridization of nucleic acid probes with complementary sequences of varying degrees of homology.

Thus, nucleic acids encoding GTS1 proteins may be used to advantage to identify and characterize other genes of varying degrees of relation to the genes of the invention thereby enabling further characterization of the molecular mechanisms controlling biomass accumulation, early flowering and increased growth development. Additionally, the nucleic acids of the invention may be used to identify genes encoding proteins that interact with GTS1 (e.g., by the “interaction trap” technique), which should further accelerate identification of the molecular components involved in biomass accumulation. A new yeast two-hybrid screen (Cytotrap) available from Stratagene, which is based on the SOS-Ras signaling pathway, is complementary to the Gal4 or LexA interaction trap system which assays for protein-protein interactions in the nucleus. In contrast, the Stratagene Cytotrap system monitors interaction in the cytoplasm, hence its name. This system, which appears to have several advantages, may be the preferred screen for GTS1-interacting proteins.

Nucleic acid molecules, or fragments thereof, encoding GTS1 genes, for example, may also be utilized to control the production of GTS1 proteins, thereby regulating the amount of protein available to participate in the induction or maintenance of biomass accumulation in plants. As mentioned above, antisense oligonucleotides corresponding to essential processing sites in GTS1-related mRNA molecules or other gene silencing approaches may be utilized to inhibit GTS1 protein production in targeted cells. Yet another approach entails the use of double-stranded RNA mediated gene silencing. Alterations in the physiological amount of GTS1 proteins may dramatically affect the activity of other protein factors involved in biological pathways regulating flowering, seed production, resistance to stress and biomass accumulation.

The nucleic acid molecules of the invention may also be used to advantage to identify mutations in GTS1 encoding nucleic acids from plant samples. Nucleic acids may be isolated from plant samples and contacted with the sequences of the invention under conditions where hybridization occurs between sequences of sufficient complementarity as described in Example 1. Such duplexes may then be assessed for the presence of mismatched DNA. Mismatches may be due to the presence of a point mutation, insertion or deletion of nucleotide molecules. Detection of such mismatches may be performed using methods well known to those of skill in the art.

Nucleic acids encoding the GTS1 proteins or mutated variants thereof may also be introduced into host cells. In a preferred embodiment, plant cells are provided which comprise a GTS1 protein encoding nucleic acid such as SEQ ID NO: 1 or a variant thereof. Host cells contemplated for use include, but are not limited to, Arabidopsis, rice, maize, wheat, tomato, potato, barley, canola, bacteria, yeast, insect and other animal cells including human cells. The nucleic acids may be operably linked to appropriate regulatory expression elements suitable for the particular host cell to be utilized. Methods for introducing nucleic acids into host cells are well known in the art. Such methods include, but are not limited to, transfection, transformation, calcium phosphate precipitation, electroporation, lipofection and biolistic methods.

The host cells described above or extracts prepared from them containing GTS1 may be used as screening tools to identify compounds which modulate GTS1 protein activity. Modulation of GTS1 activity, for example, may be assessed by measuring alterations in flowering times, seed production or biomass accumulation in the presence of a test compound. Test compounds may also be assessed for the induction and/or suppression of expression of genes regulated by GTS1 proteins.

The availability of GTS1 protein encoding nucleic acids enables the production of plant species carrying part or all of a GTS1-related gene or mutated sequences thereof, in single or amplified copies. Transgenic plants comprising any one of the GTS1-related sequences described herein are contemplated for use in the present invention.

The alterations to the GTS1-related genes envisioned herein include modifications, deletions, and substitutions. Such modifications, deletions or substitutions can result in an GTS1 having altered characteristics or functions. Alternatively, modifications and deletions can render the naturally occurring gene nonfunctional, producing a “knock out” plant. In this context, a “targeted gene” or “knock-out” is a DNA sequence introduced into the plant by way of human intervention, including but not limited to, the methods described herein. Substitution of the naturally occurring gene for a gene having a mutation results in a plant with a mutated GTS1 protein. A transgenic plant carrying a GTS1 gene is generated by direct replacement of the first plant's GTS1 gene with the second GTS1 gene. These transgenic plants are valuable for use in in vivo assays for elucidation of molecular mechanisms associated with biomass accumulation.

The following materials and methods are provided to facilitate the practice of the present invention.

### Plant Material and GTS1 Expression Profiling Analysis

Arabidopsis thaliana (ecotype Col-0) and gts1 knockout mutant (T-DNA SALK_010647) from Arabidopsis Biological Research Center (ABRC) were used throughout this work. Appropriate seeds were sown on Murashige and Skoog (1×MS) agar plates or soil and seedlings were allowed to grow under continuous illumination (120-150 μEm-2s-1) at 24° C. Seedling samples were collected at different developmental stages for gene expression profiling. To analyze the expression of GTS1 gene, total RNA was extracted with TRIzol reagent (Molecular Research Center) and then reversed transcribed using qScript cDNA Supermix (Quanta BioSciences, Gaithersburg, Md., USA) as previously described [10]. Thereafter, the cDNA was used as the template for PCR using gene-specific primers (Table 1), running 20 or 25 amplification cycles (linear range of amplification) unless otherwise noted [11]. The linear range of amplification was determined by running increasing cycle numbers and analyzing the amount of cDNA fragments. PCR fragments were separated on 1% agarose gels containing ethidium bromide. A cDNA fragment generated from ACTIN (AT3G18780) served as an internal control. T-DNA insertion in GIGANTUS1 gene was PCR-confirmed using GIGANTUS1 gene specific primers and T-DNA left border (LB) primer (Table 1). The expression of GTS1 gene in gts1 mutant background was analyzed by extracting total RNA from the gts1 homozygous line asabove described and reverse transcribed into cDNA as previously described [10], and further used in expression analysis.

### GTS1 Database Search and Phylogenetic Analysis

The Arabidopsis thaliana GTS1 cDNA sequence (AT2G47790) was obtained from the Arabidopsis-TAIR website and used to perform a nucleotide BLAST (BLASTn) search of the Oryza sativa (rice) genomic sequence on the SALK Institute RiceGE2 web interface. The rice sequence identified as having the highest degree of homology to the Arabidopsis cDNA was downloaded and translated. The translated rice sequence was then aligned to the Arabidopsis protein sequence to validate the identification of the gene. The rice cDNA sequences were then used to perform a (BLASTn) search against the sequenced Zea mays genome on the MaizeSequence.org website. BLAST searches against the maize genome produced a list of BAC sequences that aligned to the query sequence. The BAC with the highest level of similarity was indicated on a genome map. The complete BAC sequence was downloaded and aligned to the Oryza sativa cDNA sequence using the NCBI BLAST (bl2seq) algorithm. Once the putative exons had been identified for a specific gene homolog, the exon start and end positions were manually corrected based on the canonical splice site donor/acceptor sequences and the overlapping sequence from one putative exon to the next. Each putative maize cDNA was then translated into a protein sequence. The GTS1 protein sequences from both Oryza sativa and Zea mays were aligned to the Arabidopsis thaliana sequence using the EMBOSS global pairwise alignment algorithm [12] to get the percent identity between the proteins. Next a BLASTp search of the Homo sapiens proteome in the NCBI database was performed using the Arabidopsis thaliana GTS1 protein sequence. From this search the Wdr89 sequence of Homo sapiens was identified as the most homologous protein. The Wdr89 sequence was used to retrieve (BLASTp) the Rattus norvegicus and the Mus musculis sequence homologs. Finally, a ClustalW2 alignment [13] was performed aligning all of the GTS1 protein sequences from Arabidopsis thaliana, Oryza sativa, and Zea mays with the Wdr89 protein sequences from Homo sapiens, Ratus norvegicus, and Mus musculis.

The conserved WD40 protein sequence from Arabidopsis thaliana was identified using ScanProsite and the Swiss-Prot/TrEMBLE databases. The identified WD40 sequence was used to perform a BLASTp search against plant proteomes on NCBI. From the list of potential WD40 containing proteins, the cDNA sequence of several GTS1-like proteins were downloaded from Nicotiana benthamiana, Nicotiana tabacum, Pisum sativum, Phaseolus vulgaris, Medicago truncatula, Gossypium hirsutum, Lycopersicon esculentum, Solanum chacoense, Solanum lycopersicum, and Physcomitrella patens. The collected plant WD40 containing cDNA sequences were combined with the Arabidopsis thaliana, Oryza sativa, and Zea mays cDNA sequences previously obtained and aligned using RevTrans version 1.4 [14]. Using MrAIC [15-20] model test software the best AIC model to use in constructing phylogenetic relationships was determined to be the General Transition Rate (GTR) with Gamma.

Phylogenetic trees were generated in MrBayes using four Markov Chain Monte Carlo runs with three cold and one hot chain each for 1,000,000 generations sampling every 100 generations. The burn-in was determined to be within the first 4000 generations for each phylogeny. The first 10000 generations sampled were removed and a 50% consensus majority tree was constructed from the remaining trees. Trees were then drawn using TreeView version 1.6.6 and rooted using the Physcomitrella patens sequence as an out group.

### Sequences Interaction Database Search

Arabidopsis thaliana GTS1, a Transducin/WD40 repeat protein (NCBI accession number AEC10888) was used as query to retrieve WD40 protein sequences from Uniprot (www.uniprot.org), and NCBI (www.ncbi.nlm.nih.gov) databases using BLAST tools (blast.ncbi.nlm.nih.gov/Blast.cgi).

GTS1 protein interaction network was obtained using the STRING v9.0 (string-db.org) database. STRING outcome gave the two most possible interacting protein counterparts for GTS1 protein, a 60S ribosome structural protein L 19e (NCBI accession number AEE75864), and Nop16 (NCBI accession number AAP21378), a protein involved in 60S subunit ribosomal biogenesis.

### Functional Domains Analyses

GTS1 protein functional domains were studied by querying different structure-functional motifs and/or patterns databases such as Pfam v25.0 (pfam.sanger.ac.uk), Prosite (prosite.expasy.org/scanprosite), SMART v6.0 (smart.embl-heidelberg.de), Conserved Domain Database (CDD) v3.02, CDART (Conserved Domain Architecture Retrieval Tool) and CD-Search tools on the world wide web at ncbi.nlm.nih.gov/Structure/cdd/cdd.shtml), InterPRO v35.0 on the world wide web at .ebi.ac.uk/interpro), ProDom release 2010.1 (prodom.prabi.fr/prodom/current/html/home.php), CATH v3.4 on the world wide web at cathdb.info), Superfamily v1.75 (supfam.cs.bris.ac.uk/SUPERFAMILY), PIRSF (pir.georgetown.edu), and functional searched by PANTHER on the world wide web at .pantherdb.org. Similar analysis was performed for both interacting ribosomal protein counterparts, Nop16 and L19e.

### Secondary Structure Prediction

Secondary structural elements of the GTS1 protein were initially assessed for substructure conserved motifs by threading the sequences through the Protein Data Bank (PDB) on the world wide web at pdb.org library using threading algorithm the Segmer[21]. These elements of the secondary structure were also confirmed by comparison with the results obtained with other additional 2-D structure servers: SSpro8 (Scratch Protein Predictor, scratch.proteomics.ics.uci.edu), NetSurfP ver. 1.1 on the world wide web at cbs.dtu.dk, and PSIPRED (bioinf.cs.ucl.ac.uk/psipred) fold servers. These secondary structure elements prediction were also performed for both interacting ribosomal protein counterparts, Nop16 and L19e.

### Structural Templates Searching

Protein sequences of GTS1, Nop16 and L19e were searched for homology in the Protein Data Bank (PDB). Homologous templates suitable for these three proteins were selected by BLASTp from the BLAST server (ncbi.nlm.nih.gov). The BioInfoBank Metaserver (meta.bioinfo.pl), which employs fold recognition for homology search, was also used for the selection of templates. The results obtained by previous methods were compared with the results obtained by Swiss-model server for template identification (swissmodel.expasy.org).

### Homology Modeling

Homology modeling was performed by the I-TASSER server [22]. An initial structural model was generated and checked for recognition of errors in 3D structures using ProSA (prosa.services.came.sbg.ac.at/prosa.php), and for a first overall quality estimation of the model with QMEAN (swissmodel.expasy.org/qmean/cgi/index.cgi).

Energy minimization of the final structures was performed using GROMOS96 force field energy implemented in DeepView/Swiss-PDBViewer v3.7 (spdbv.vital-it.ch) in order to improve the van der Waals contacts and correct the stereochemistry of the model.

Each structure was assessed using the following softwares: QMEAN for quality, PROCHECK on the world wide web at ebi.ac.uk/thornton-srv/software/PROCHECK) for stereological corrections, ProSA, and ANOLEA (protein.bio.puc.cl/cardex/servers/anolea) for protein energy. The number of protein residues in the favored regions for each structure were calculated and visualized by The Ramachandran plot.

### Ligand-Binding Domains and Conservational Analysis

Ligand-binding sites in the 3D protein structures were analyzed using Cofactor software (zhanglab.ccmb.med.umich.edu/COFACTOR), to identify functional homology. Gene Ontology (GO) terms (The Gene Ontology project) were used to identify functional analogs based on the 3D built models, indicating the possible functions and biological pathway in which the proteins might be involved (See the world wide web at geneontology.org).

Conservational analyses of the proteins were made by generating evolutionary related conservation scores using ConSurf server (consurf. tau.ac.il). Structural function conservation and key residues in the query proteins were identified by ConSeq server (conseq.tau.ac.il).

### Surface Electrostatic Potential Analysis

The electrostatic Poisson-Boltzmann (PB) potentials for the surface amino acids of the structures were obtained using APBS software implemented in PyMol 0.99 on the world wide web at pymol.org, with AMBER99, and optimized with the Python software package PDB2PQR. Fine grid spaces of 0.35 Å were used to solve the linearized PB equation in sequential focusing multigrid calculations in a mesh of 130 points per dimension at 310.00 K. The dielectric constants were two for the proteins and 80.00 for water. The output mesh was processed in the scalar OpenDX format to render the isocontours and maps on the surfaces with PyMOL 0.99. Potential values are given in units of kT per unit charge (k Boltzmann's constant; T temperature).

### Molecular Docking Analysis

The analysis of interactions between GTS1 with each of its ribosomal counterparts (Nop16 and L19e,) were performed using CLUSpro server [23]. During the workflow, backbone flexibility analysis was done using rigid-body ensemble docking with multiple structures derived from NMR, while the ZDOCK option for sampling at 6-degree rotational steps was used to obtain the decoys. The energies of docked conformations from protein-protein docking were evaluated by applying the Fast Fourier Transform (FFT) correlation approach.Docking scores were calculated by considering shape complementarity, desolvation, and electrostatics potential. The top docked conformations, along with their ZDOCK scores, were used as candidates of near-native structures, and for clustering of binding sites where the ligand was within 10 Å of its receptor.

After clustering, the ranked complexes were subjected to van der Waals minimization using CHARMM, and the protein-inhibitor structure with the best score was chosen as the most fitting model for GTS1-ribosomal interacting proteins.

The following examples are provided to illustrate certain embodiments of the invention. They are not intended to limit the invention in any way.

### Example I

### Molecular and Structural Analysis of GTS1

### Expression Profiling and Phylogenetic Analysis of GTS1

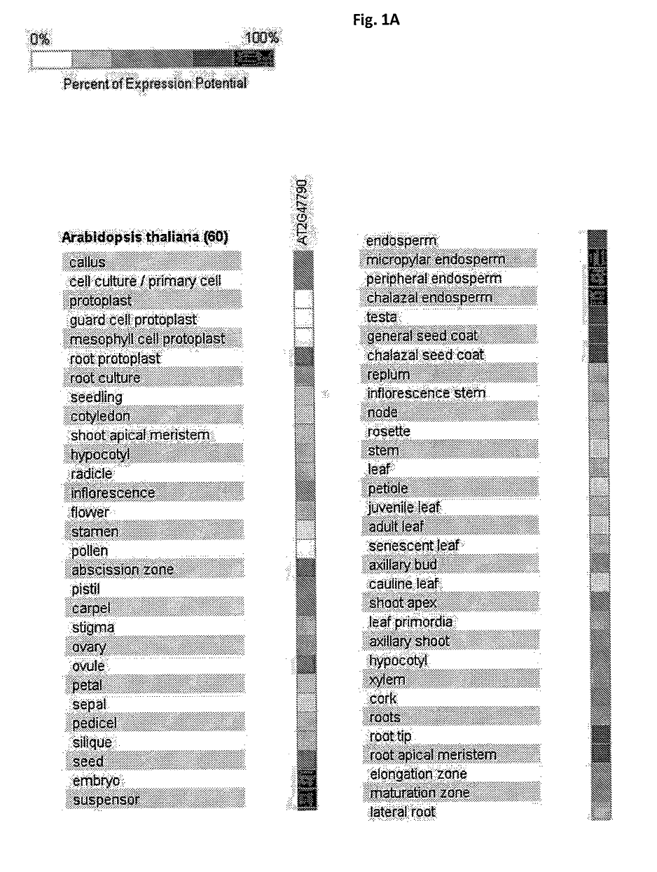

The Arabidopsis GTS1 is highly expressed during seed germination, particularly accumulating in embryo, ovule, and endosperm, (FIG. 1A, FIG. 1B). It is abundantly expressed in meristemic regions, indicating its crucial role in regulating cell divisions (FIG. 1A). The strong tissue (abscission zones) specific expression pattern of GTS1 (FIG. 1A) suggests its regulatory implication in plant growth developmental process (FIG. 1A, FIG. 1B).

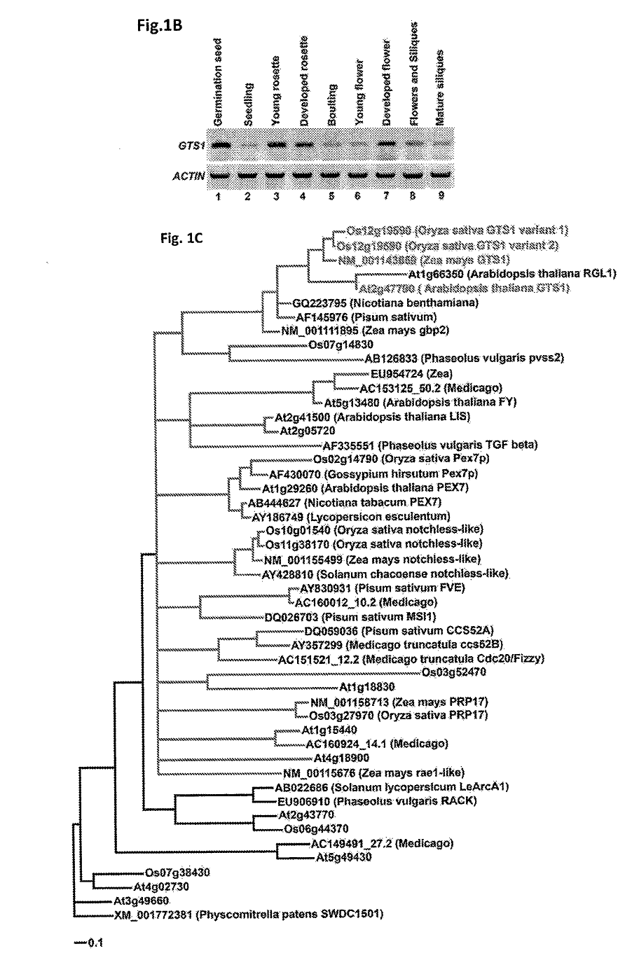

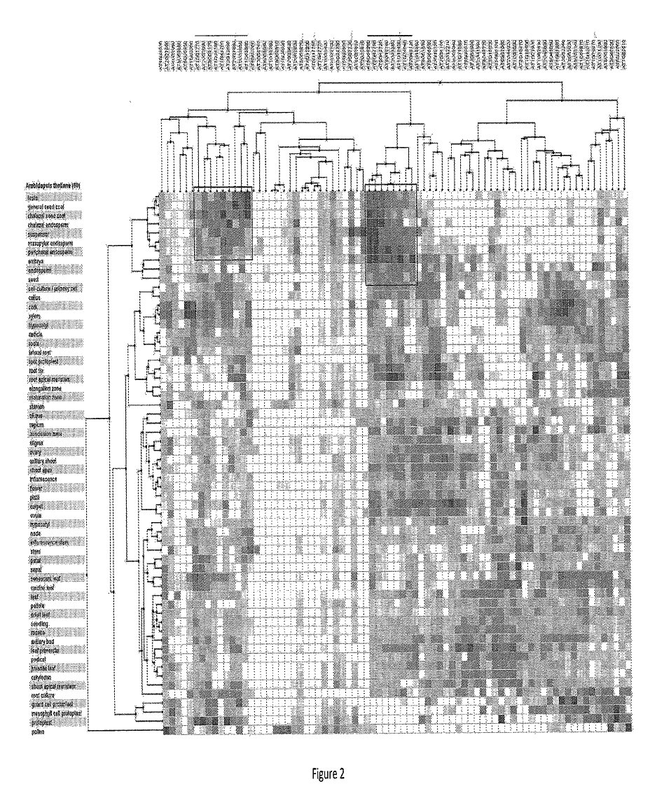

We confirmed that GTS1 transcript accumulates in several major organs, including developing flowers, germinated seeds, and young rosette leaves (FIG. 1B). Microarray data analysis at different developmental stages also reveals an overlapping expression pattern of cell division/growth induced genes with GTS1 (FIG. 2) as highlighted in Table 2 [24]. These genes are involved in transcriptional and posttranscriptional processes, and various biochemical pathways (Table 2). On the basis of the high level of amino acid identity between Arabidopsis GTS1 and other well characterized WD40 protein homologs (FIG. 3), a phylogenetic analysis showed that Arabidopsis GTS1 is clustered with the rice and maize GTS1 (FIG. 1C), thus indicating that they are more similar to one another than they are to other GTS1-WD40 repeat sequences (FIG. 1C). This cluster belongs to a subclade of the dominant Glade (FIG. 1C, gray branches) containing most of the plant GTS1 protein homologs.

### GTS1 Regulates Seed Germination and Plant Growth Development

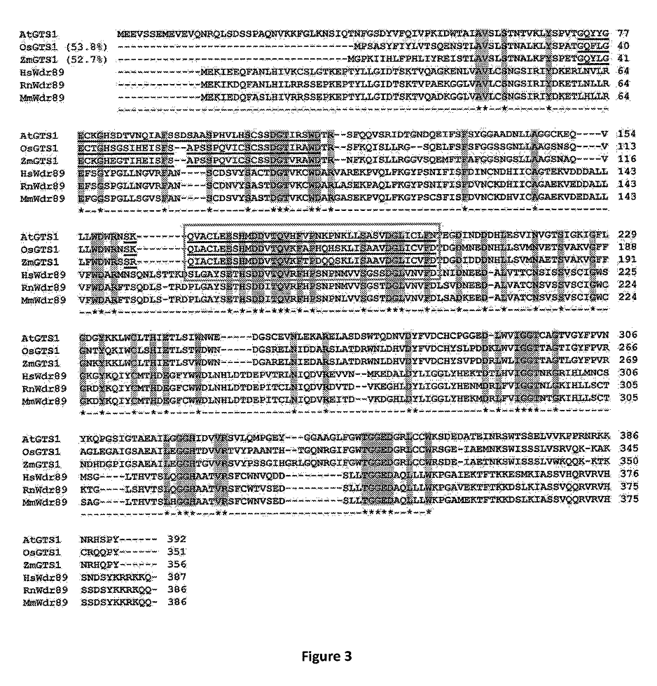

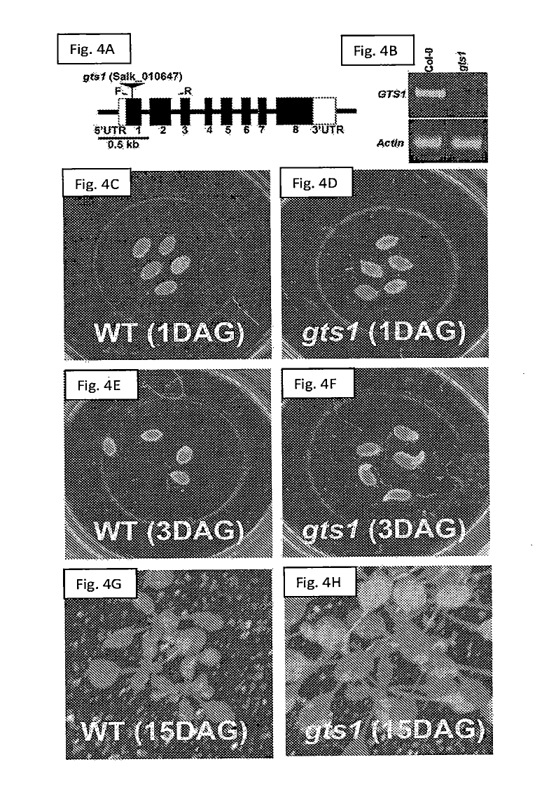

In order to examine the role of GTS1 in plant growth development, we employed a reverse genetic approach using a SALK_TDNA knockout insertion (Salk_010647) of GTS1 gene (FIG. 4A) to investigate the effect of loss of GTS1 function in gts1 mutant. The SALK_010647 (gts1) line harbored a T-DNA insertion in the first exon of GTS1 gene (FIG. 4A), which was PCR-confirmed by using the T-DNA-specific oligonucleotide primer LB1 and the GTS1-specific primers (Table 1). We next examined and confirmed the knockout GTS1 mRNA transcript levels in gts1 compared to WT using RT-PCR (FIG. 4B). When compared to the wild type, the homozygous gts1 mutant (n=16) displayed a faster germination rate (FIG. 4C-FIG. 4F), and a faster growth rate and higher biomass accumulation than the wild type (n=16) (FIG. 4G, FIG. 4H), indicating that GTS1 negatively regulates cell division, growth and overall biomass accumulation in meristemic regions. Furthermore, gts1 mutant (n=22) flowers earlier (5 days earlier) than the WT (n=16) (FIG. 5A), as demonstrated by a reduced number of rosette leaves (9±0.6, n=15) compared to the wild type (15±0.5, n=20) at bolting time (FIG. 5B). The mutant (gts1) grows significantly taller than WT at the same day post germination (FIG. 5A).

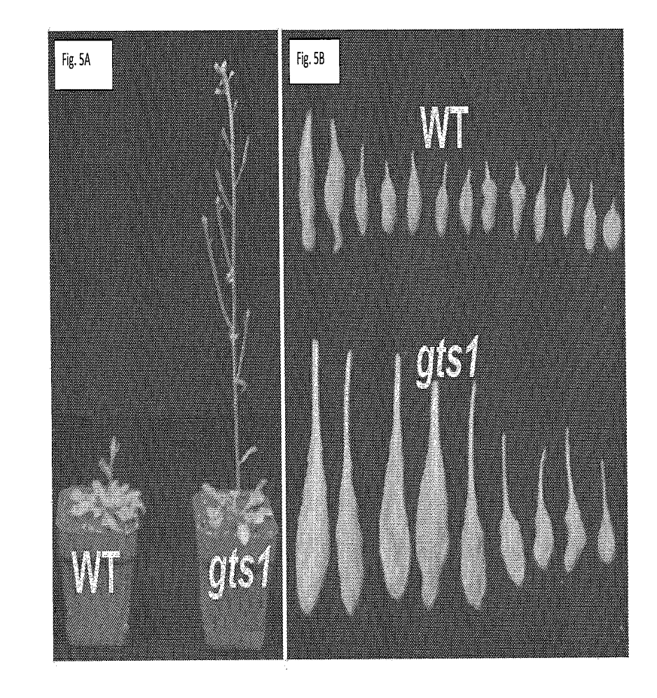

In order to confirm that GTS1 is indeed responsible for these phenotypes, we performed a complementation test by RT-PCR amplifying a 1095bp of GTS1-encoding sequence from WT cDNA (Table 1), cloned it into the Smal site of the pROK2 vector [25] under control of a CaMV 35S promoter to drive overexpression [26] and stably transformed gts1 mutant background by the floral dip method [27]. As expected, the overexpression of GTS1 in gts1 mutant background was sufficient to abolish the above described gts1 phenotypes. The complemented line displayed a WT-like phenotype, indicating that this GTS1 is indeed responsible for the phenotypic characterization in gts1 mutant. Unlike the other co-expression gene patterns (Table 2), we interestingly identified a strong gene-to-gene functional relationship between GTS1 (AT2G47790) and the ribosomal protein L19e (At3g16780) (FIG. 6). This data shows the detail of stability of co-expression between the 2 genes. The co-expression was supported by many factors, where the PCA correlation remained unchanged regardless of the growth parameter/factor considered. Both the x and y axis are relative gene expression values in base-2 logarithm against the averaged expression levels of each gene (FIG. 6). These data argue for a strong gene-to-gene functional gene expression, suggesting that these two genes/proteins physically interact to regulate/control biological processes in plants.

TABLE I

| Name | Primer Sequence | Description |
|---|---|---|
| GTS1-F1 | 5′GAGGAGCTGCAGGGTTATTT3′ (3)* | For RT-PCR |
| GTS1-R1 | 5′CAAGACGGGTTAATCTGGGTAG3′ (4) | For RT-PCR |
| TDNA-LB | 5′CCGTCTCACTGGTGAAAAGAA3′ (5) | For TDNA insertion |
| GTS1-F2 | 5′CTGAAACGGCAAATGGAAGAAG3′ (6) | For complementation test |
| GTS1-R2 | 5′CTATGTTGCTGGAAGTCGGAT3′ (7) | For complementation test |
| Act2-F | 5′GCGGATCCATGGCTGAGGCTGATGATATTCAACC3′ (8) | For RT-PCR |
| Act2-R | 5′CGTCTAGACCATGGAACATTTTCTGTGAACGATTCC′ (9) | For RT-PCR |

*numbers in parenthesis are SEQ ID NOS:

TABLE 2

| Gene Locus | Function |
|---|---|
| AT2G47790 | Transducin/WD40 repeat-like superfamily protein |
| (GTS1) | |
| At3g13460 | evolutionarily conserved C-terminal region 2 |
| At5g04290 | kow domain-containing transcription factor 1 |
| At1g43700 | VIRE2-interacting protein 1 |
| At1g01770 | Protein of unknown function DUF1446 (InterPro:IPR010839) |
| At5g43720 | Protein of unknown function (DUF2361) |
| At1g74040 | 2-isopropylmalate synthase 1 |
| At2g32850 | Protein kinase superfamily protein |
| At2g22090 | RNA-binding (RRM/RBD/RNP motifs) family protein |
| At3g52120 | SWAP (Suppressor-of-White-APricot)/surp domain- |
| | containing protein/D111/G-patch domain-containing protein |
| At3g16780 | Ribosomal protein L19e family protein |
| At2g48120 | pale cress protein (PAC) |
| At2g21580 | Ribosomal protein S25 family protein |
| At1g34180 | NAC domain containing protein 16 |
| At2g27880 | Argonaute family protein |
| At1g62990 | KNOTTED-like homeobox of Arabidopsis thaliana 7 |
| At1g16430 | Surfeit locus protein 5 subunit 22 of Mediator complex |

### Searching for Structural Templates

Since we confirmed that GTS1 regulates seed germination and growth development (biomass yield, and flowering time) in plants (FIG. 4, FIG. 5), we next examined the protein structure scaffold and interacting partners of GTS1 which play a role on these functions. In order to study the physical interactions of GTS1 with other proteins in regulating growth development in plants, we performed a Protein Data Bank (PDB) search for GTS1 protein with known tertiary structure in PDB. The search yielded the crystal structures/PDB accession numbers 2h9l, 3iz6, 3ow8, 2gnq, and 1tbg, showing the highest sequence identity (22, 17, 16, 24, and 15%, respectively). The suitability of selected model was checked by BioInfoBank Metaserver, which returned 3D Jury scores (J-score) of 208.4 (2h9l), 200.2 (3iz6), 198.5 (3ow8), 205.7 (2gnq), and 201.9 (1tbg) for GTS1, respectively. In order to confirm the best possible templates to use for building the GTS1 structure, a Swiss-Model server was used, finding high scores (64, 61, 63, 60, and 69) and very low E-values (1.5×10⁻³⁷, 2.1×10⁻³⁰, 4.9×10⁻²⁹, 2×10⁻¹⁰, and 1.8×10⁻⁴³) for the templates 2h9l, (3iz6), 3ow8, 2gnq, and 1tbg respectively.

The same workflow was followed to obtain the best crystal templates in order 3D-structural protein model for the GTS1 ribosomal interacting fpartners. The search in the Protein Data Bank (PDB) for the protein L19e and Nop16 yielded the crystal structures of 3iz5, 3jyw, and 3u5e for L19e, and 2aje, 1w0t, 2juh, 2ckx, and 2roh for Nop16, showing comparable values in identity and suitability by BioInforMank Metaserver and Swiss-Model server analysis.

### Quality of Threading Models

To assess the quality of the protein models I-TASSER and Procheck analysis were performed. The I-TASSER analysis gave the accuracy parameters such as a C-score of −0.9, 0.60±0.14 TM-score with 1848 decoys and 0.1467 of cluster density for GTS1, while Procheck analysis revealed that the main chain conformations of GTS1 protein model were located in the acceptable regions of the Ramachandran plot. A majority of residues (77.6%) were in the most favorable regions, whereas 14.5% of the residues were placed in the allowed regions, and 6.5% were in generously allowed regions. Only 1.4% of the residues were present in the disallowed regions, respectively. The plot of x1 versus x2 torsion angles for each residue showed that most of the rotamers in GTS1 model was localized in low energy regions. iii) The ProSa analysis gave Z-scores of −5.71 for GTS1. The scores were within the range usually found for native proteins of similar size, i.e., −7.31, −4.02, −6.63, −7.93, and −7.33 for the templates 2h9l, 3iz6, 3ow8, 2gnq, and 1tbg crystal structures, respectively. iv) QMEAN analysis of GTS1 model revealed Q-values of 0.67. A quality factor of 0.793, 0.315, 0.71, 0.786, and 0.849 was estimated for the crystal structures of the templates 2h9l, 3iz6, 3ow8, 2gnq, and 1tbg, respectively, indicating that the GTS1 model is within the range of accuracy of the templates crystallographic structures. v) Root mean square deviation (RMSD) between GTS1 model and the crystal templates Ca backbones of the closed templates were 2.408 Å for 2gnq and 3.192 Å for 2h9l.

All of the above parameters were also determined for L19e and Nop16 protein models resulting in comparable structural quality values of our modeled L19e and Nop16 proteins.

3D structure of Arabidopsis GTS1

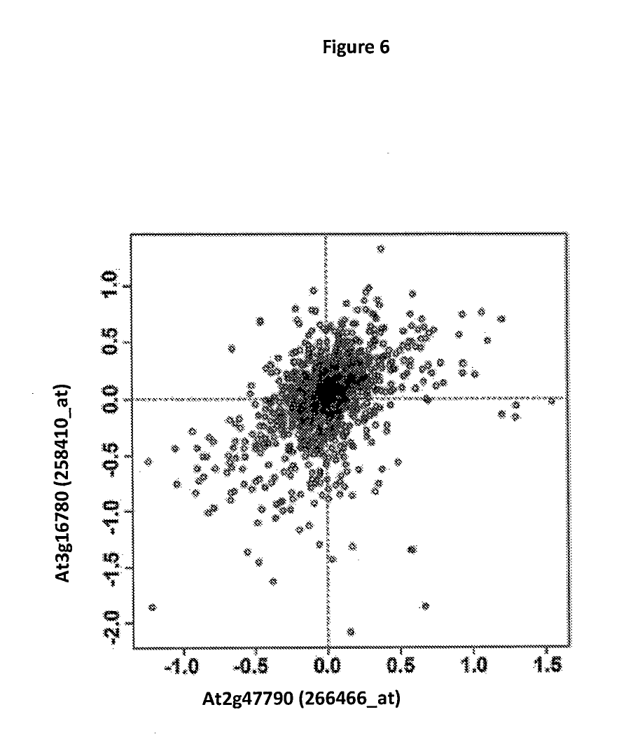

We obtained the best structural models of this newly described Arabidopsis WD40 repeat protein, GTS1, based on homology modeling (FIG. 7). The 3D structure of Arabidopsis GTS1 belongs to the transducin/WD40 repeat protein family because it shares all the structural characteristics of WD40 proteins, (FIG. 7A), which agree with the general crystal structure of 2h9l or 2gnq template. In general, the structure can be visualized as a short, open cylinder where the strands form the walls. At least four repeats are required to form a β-propeller. In our case, GTS1 contains 7 WDs, where the final and last (i.e., the N- and C-terminal) WDs participate in the same β-propeller (FIG. 7A), potentially reinforcing the structure. Despite the low amino acid sequence identity across species, a relatively good conservation of the overall fold (Cα carbon chain) of this protein is found among plant species and eukaryotes in general [4, 28].

Surface electrostatic potential analysis (FIG. 7B) reveals several prominent positively charged residues (blue regions), predominantly in the walls of the cylinder (tunnel) and C-terminal arm. The environment of this protein is essentially negatively charged (red regions) (FIG. 7B), as highlighted by the Poisson-Boltzmann electrostatic potential. By assigning a value of +1 to basic residues (R, K) and −1 to the acidic residues (D, E), net charge of protein was calculated to be -22 for GTS1. The central tunnel of the GTS1 structure exhibited a predominantly positive charge in the top view. Comparison between GTS1 and other WD40 repeat proteins, such as templates 2gnq and 2h9l did not exhibit large differences in the general topology as it was further confirmed by the RMSD value of 2.408 Å and 3.192 Å, respectively, whereas significant differences were found in particular regions of the proteins such as the N-terminal, and C-terminal regions.

### Conservational and Functional/Ligand-Binding Site Analysis

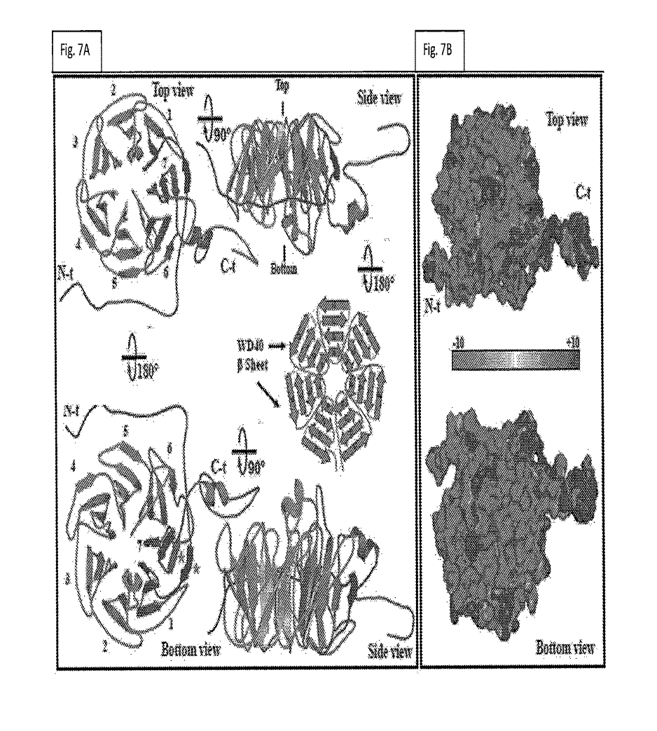

The conservational and ligand-binding or functional features of GTS1 were analyzed (FIG. 8). Surfaces of Arabidopsis GTS1 (rotated)180° showing the conservation index of residues are depicted in FIG. 8A. Consurf conservation analysis showed that GTS1 cylinder-like β-propeller structure is quite well conserved, especially the residues located in the core of the structure (FIG. 8A). The N- and C-terminal regions of the protein are also highly conserved regions of the protein, besides the core area which has a major role in maintenance of the protein structure [9, 29]. This distribution of the core conserved region and surface variable residues helps maintain a similar overall fold among WD40 repeat proteins, but also produces differences observed in terms of a multiple protein interactions along WD40 repeat protein, where it is indicated by a discontinued red line for both ribosomal proteins in the GTS1 structure (FIG. 8A).

FIG. 4B shows a general view of the amino acids in the tunnel-like structure holding up the interacting peptides. GTS1 exhibit the putative active (most representative ligand-binding) site located in the center of the structure (FIG. 8B, top view), which normally is another interacting polypeptide, containing several conserved but also few variable amino acids. A detailed view, showing the spatial distribution of residues responsible for the conformation of the ligand binding domain surrounding the ligand and directly implicated in this interaction are Y43, V44, F45, S61, N87, S107, F134, V194, 5267, R329, and the peptide chain substrate bound to the tunnel-like structure (FIG. 8B, detailed view at the right side). Conservational analysis of WD40 repeat proteins with significant close identity to Arabidopsis GTS1 among other species returned a large number of highly variable (bold) and small number of conserved (italic) residues as written above. Conformational predictions indicate that the peptide (ligand) is projected to and partially located inside the tunnel-like structure of GTS1.

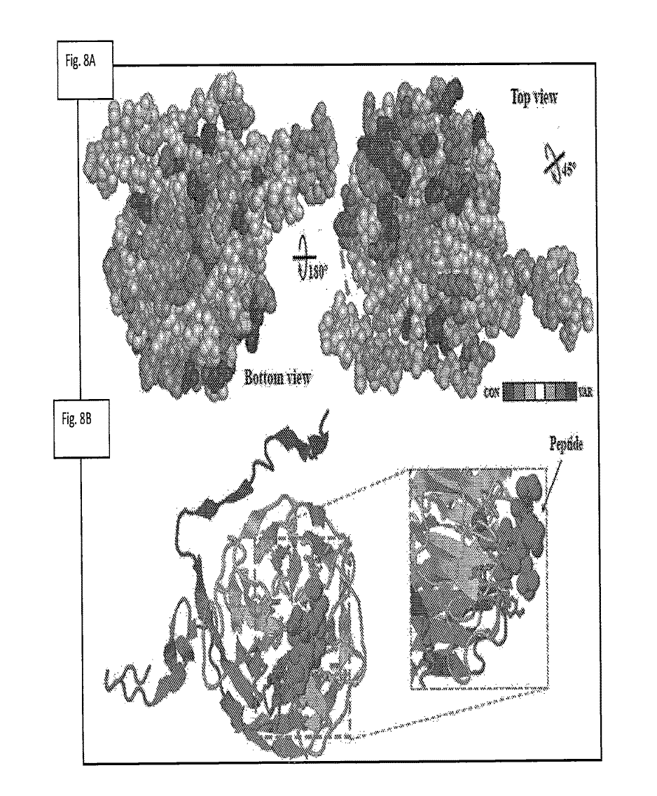

The conservational and functional analysis of the ribosomal L19e and Nop16 proteins are depicted in the FIG. 9. Consurf conservation analysis in the FIG. 8A showed that Nop16 protein is well conserved, especially in the interacting surface region (red arrows, and FIG. 9B) and the nucleic acids (blue arrow). Only few light and deep blue residues are around the surface of the protein. Ligand-binding analysis of this protein showed in FIG. 9B highlights the area of the Nop16 protein where the interaction with nucleic acids takes place. This area is located in a cleft integrated by the N-terminal α-helix. The residues implicated in the interaction between the protein and the nucleic acid are L40, M41, T142, and R146, resulting in four not well conserved amino acids (italic)

Consurf conservation analysis showed in the FIG. 9C highlights an equally distributed number of conserved and variable residues along the surface of L19e protein. The area of interaction with GTS1 seems not to be as well conserved as its counterpart Nop16. Few variable residues (blue color) are located in the area of interaction with the N-terminal tail of GTS1 (red arrows in FIG. 9C). In addition, among the surface directly implicated in the interaction with the ribonucleic acid (blue arrows in FIG. 9C, FIG. 9D), S3, K5, I6, R9, L10, N36 seems to be well conserved, since only one residue, I6, was found to be variable (italic), and the rest exhibit an average index of conservation or highly conserved like R9 (bold).

GTS1 Interaction Mechanism: Molecular Docking Analysis with Ribosomal Counterparts

In order to get insights into the GTS1 regulatory/multi-interacting mode with other proteins, we analyzed the conformational interaction between GTS1 and two interacting partners involved in the structure and biogenesis of ribosomes in Arabidopsis. This analysis was carried out by molecular docking, using newly modeled structures of the two ribosomal proteins.

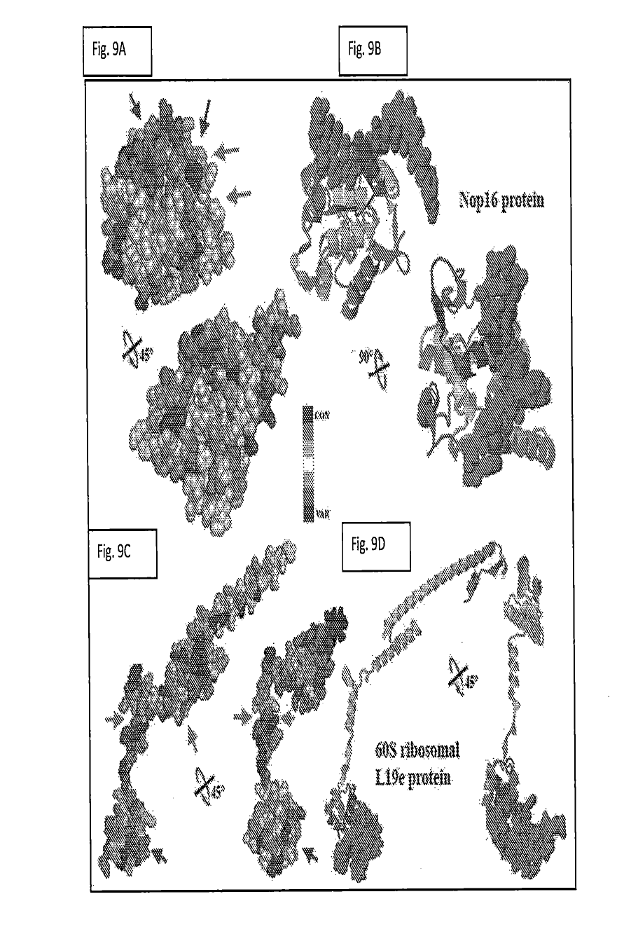

FIG. 10 shows the mode of interaction between Arabidopsis GTS1 and the nucleolar protein 16 (Nop16), involved in the biogenesis of 60S ribosomal subunit. The binding mechanism occurs through the formation of a stoichiometry complex 1:1 between both proteins. A detailed view of this interaction is depicted in the magnified views displayed in FIG. 10A, where C-terminal arm of GTS1 is located inside the cavity made by N-terminal α-helix of Nop16 and neighboring helices, in addition to the direct interaction of this N-t α-helix with the β-propeller 4 structure of GTS1. The N-t α-helix of GTS1 is covered completely by the Nop16 cleft, therefore preventing access by other interacting partners to this area. On the other hand, it also partially impedes the interactions that are necessary for binding the nucleic acid to Nop16 by stereological impediment. The interaction is non-covalent, thus it may be reversible by increasing salt concentration and/or pH to alkaline conditions. FIG. 10B shows the large area of interaction covered by the Nop16 molecule in the GTS1 in a perpendicular (90°) view.

With regard to energy, the electrostatic potential analysis for the contact surface of both structures exhibited a highly compatible fingerprint distribution of opposite charges, i.e., positive (in the contact area of Nop16 protein), and negative (mainly for GTS1) (FIG. 10C). There are not large areas with hydrophilic character in the contact surface between both proteins, and the formation of the complex may be mediated by a high number of direct and water-mediated H-bonds.

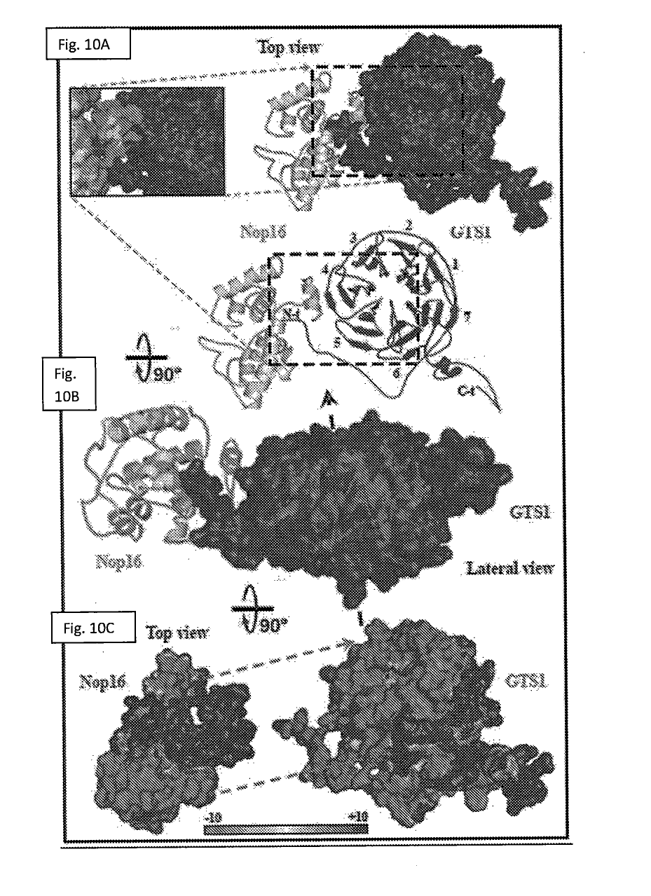

FIG. 11 shows the mode of interaction between Arabidopsis GTS1 and the 60S ribosomal protein L19e. The binding mechanism also occurs through the formation of a stoichiometric complex 1:1 between both proteins. A detailed view of this interaction is depicted in the magnified view (FIG. 11A), where the N-terminal area of GTS1 hugs the thin structure of L19e, and the neighboring α-helix contacts the β-propeller 4 of GTS1. Additionally, the N-terminal area of the L19e protein directly interacts with the residues of the central bottom side of the GTS1 tunnel, leaving the central top side of the tunnel free as detailed in FIG. 10B. The same area of L19e protein is also involved in the interaction with ribonucleic acid (FIG. 9D). The electrostatic potential analysis for the contact surfaces of both structures exhibited a highly compatible fingerprint of opposite charges (positive in the contact area of L19e protein and negative in the contact surface of GTS1) (FIG. 11C).

### Discussion

GIGANTUS1 is here described to be very important in regulating plant growth development (seed germination, faster growth, flowering time, and biomass accumulation) (FIG. 1, FIG. 4 and FIG. 5). This is the first time that a mutation in GTS1 has been implicated in early germination, growth and development in Arabidopsis thaliana. The molecular mechanism by which GIGANTUS1 regulates plant growth development is still unknown. As a member of WD40 protein family, GTS1 is expected to play central roles in different biological processes including cell division and cytokinesis, flowering, floral development, cytoskeleton dynamics, nuclear export to RNA processing, transcriptional mechanism, and protein-protein interactions [4]. We postulate that GIGANTUS1 might primarily function as a site for protein-protein interaction or mediator of transient interplay among other proteins to regulate different biological processes in plants. The development of protein complexes involves regulatory interactions that are mainly controlled by scaffolding proteins, such as WD40 repeat motifs. These motifs are important features of diverse protein-protein interactions [4], providing an unbending platform for interactions of proteins with other cellular components and controlling therefore several vital functions of the cell, such as signaling cascades, cellular transport and apoptosis [29-31].

The WD40 domains in GTS1 protein are shown to contain seven or multiples of seven repeats forming a highly stable β-propeller structure (FIG. 7A). The 7-fold β-propeller is the most stable β-sheet geometry characterizing the resolved WD40 structures and also used to identify WD40 proteins [32]. However members of this protein family have been also found to contain as high as sixteen repeats [33]. Proteins with less than 7 repeats form an incomplete β-propeller structure and require additional WD-repeats from their neighbors to stabilize themselves, making dimers [34]. There is no apparent folding order for each repeat and the order in which repeats fold might vary among different WD40 proteins, or even within the same protein [35]. These proteins are known be involved in light signaling/photomorphogenesis and flowering [36], auxin response and cell division [37], in meristem maintenance [38], floral development [39], seed development and flowering [40], chromatin-based gene silencing and organogenesis [39], protein turn-over, microtubule dynamics, phospholipid binding and vesicle coating [4]. This justifies the great deal of research interest in the WD40 protein superfamily across plant species. Our data (FIG. 4, FIG. 5) suggest that GTS1 belongs to the WD40 protein subfamily regulating auxin response and cell division [37], meristem maintenance [38], floral development [39], seed development and flowering [40]. In this study the Arabidopsis GTS1 ligand-binding domain (main functional domain) lies mainly on the top surface residues, which integrates parts of β-propeller domain (FIG. 8B). However, our data revealed that GTS1 WD40 propellers have three distinct surfaces available for interactions: the top region of the propeller, the bottom region (FIG. 11), and the circumference [4, 5] (FIG. 10 and FIG. 11), suggesting the multi-functional properties of GTS1 protein through protein-protein interactions. Indeed, protein-protein and protein-peptide interactions involved the entry site of the central channel of the β-propeller (FIG. 8B), where the majority of interaction partners (including small molecules) bind [5]. N- or C-terminal extensions of GTS1 run parallel to the tunnel-like structure, which form the complete 7 WD40 repeat domains, making them accessible for interaction with other partners (FIG. 10 and FIG. 11). WD40 domains can thus act as large interaction platforms for multiple protein interactions. In comparison to other domains, the proteins containing WD40 motif are components of several interaction pairs [41, 42], and act as scaffolds for larger complex assemblies. This work represents the first time the 3D-structural and molecular features of the GTS1 protein in plants have been examined.

To better understand what area of the GTS1 plays an important role in complex formation for functional conservation across plant species, we identified two fundamentally conserved regions; the first, located on the top rim, constituted by the residues composing blades 4 to 7, including N- and C-terminal arms; and the second large conserved surface is located on the bottom of the propeller and is mainly composed of blades 1, 2 and 3 (FIG. 8A). These regions represent potential protein-protein interaction sites [43].

In general, plant and particularly Arabidopsis-WD repeat proteins are strongly conserved. Most of these proteins are components of basic cellular machinery regulating plant-specific processes. An interesting question arises as to how these proteins evolved into their specific cellular roles. One of the key functional processes of WD proteins is the biogenesis of eukaryotic ribosomes, a highly regulated and dynamic process that begins in the nucleolus with transcription of rRNA precursor (pre-rRNA) and rapidly packaged into the 90S ribonucleoprotein particle containing ribosomal proteins, non-ribosomal proteins, and snoRNA-containing ribonucleoprotein particles (snoRNPs). The 90S pre-RNPs are converted into 43S and 66S ribosome assembly intermediates, which ultimately give rise to mature 40S and 60S ribosomal subunits [44]. It is well known that ribosome biogenesis is driven by a large number of pre-ribosomal factors that associate with and/or dissociate from the pre-ribosomal particles along the maturation pathway. Although there has been much progress to identify ribosome assembly intermediates and their protein and RNA constituents [45], the information about the architecture of these pre-rRNPs is scarce. It is unclear which proteins are the nearest neighbors within the assembled ribosomes and to what extent neighboring molecules function together.

WD40 protein-protein interaction motifs represent excellent candidates for mediating interactions in the multiprotein subcomplex required for assembling ribosomes because of their protein-protein multi-interacting versatility. More than 70 trans-acting factors required for ribosome assembly have been identified [46], as well as 80 additional assembly factors present in pre-ribosomes [47]. Therefore, such WD40-containing proteins may nucleate the assembly of pre-ribosomes by interacting sequentially or simultaneously with other assembly factors or ribosomal proteins. Among the assembly factors, 17 proteins were found to contain WD40 motifs [48]. Many of the annotated ribosome biogenesis-WD40 repeat proteins were shown to directly interact with, or regulate the levels of other proteins [49] or to be components of multiprotein subcomplexes. Yeast WD40 protein Ytml is a constituent of 66S pre-rRNPs, whose depletion resulted in a deficiency of 60S ribosomal subunits [50]. Its homolog, mammalian WDR12, functions in the maturation of the 60S ribosomal subunit. WDR12 forms a stable complex with a novel member the nucleolar proteins Pes1 and Bop1 (Pe-BoW complex), which are crucial for processing of the 32S precursor ribosomal RNA (rRNA) and cell proliferation [36]. Interestingly, a potential homologous complex of Pes1-Bop1-WDR12 in yeast (Nop7p-Erb1p-Ytm1p) is involved in the control of ribosome biogenesis and S phase entry [51].

The yeast WD40 repeat protein Mak11 that modulates a p21-activated protein kinase function is an essential factor in nuclear maturation of 60S ribosomal subunits and its depletion led to a cell cycle delay in G1, indicating an early step nucleolar role of Mak11 in ribosome assembly. Another sub-complex, transiently associated with late, nuclear pre-60S precursors, is composed of four proteins and contains Ipi3 as a WD40 repeat member [52].

In this study, a new interacting counterpart, the Arabidopsis Nop16 protein was identified as a potential ribosome biogenesis factor in plants, which could be implicated in formation of the 60S ribosomal precursor. This process appears to be regulated by an interaction with GTS1 (FIG. 10). This interaction was studied using a docking analysis that showed a stable interaction between GTS1 and Nop16, involving the N-terminal tail and the 4^{th} blade of the first partner, and a cleft formed in the second ribosomal factor by the N-terminal α-helix and the neighboring secondary elements. Another ribosomal protein (L19e protein) was found to interact with GTS1. L19e protein is implicated in the structural stability of ribosome. The interacting area between GTS1 and L19e is very close to that of Nop16 and GTS1 interacting area (FIG. 10). This suggests that the interacting mechanism of regulating the biogenesis nucleolar factor Nop16 and the structural ribosome factor L19e may be competitive. Therefore both steps in the 60S ribosomal subunit formation, structural maturation and stabilization may be separate in the time and/or different cellular compartments.

In support of our data are two other examples of WD40 repeat ribosome biogenesis factors, Rrb1 and Sqt1, which interact directly with ribosomal proteins for 60S ribosomal subunit assembly. Rrb1 interacts with the ribosomal protein Rp13 in the nucleus and regulates its levels [53], and Sqt1 interacts with Rp110 in the cytoplasm [54]. Both proteins have a role in the association of the corresponding ribosomal protein with the nascent 60S ribosomal subunits and might regulate the levels of the corresponding ribosomal protein. Other WD40 repeat proteins have been implicated in the formation and stabilization of the small ribosomal subunit 40S. Yeast RACK1 regulates the translation initiation by recruiting PKC to the ribosome [55, 56]. Four RACK1 orthologs identified in Arabidopsis thaliana may have a similar activity [57]. These interactions could provide a mechanism to regulate translation activities of ribosome populations programmed with specific mRNAs [58].

The present study demonstrates that GIGANTUS1 plays a pivotal role in controlling seed germination, faster growth and biomass accumulation in plants. The gene is mainly expressed in meristemic regions and is therefore important in cell division. The encoded protein appears to regulate growth development through diverse protein-protein interactions, including those involved in scaffolding and dynamic multi-subunit complexes such as the ribosomal protein biogenesis, stability and activity. Given its rich interaction surfaces, GTS1 likely functions in plants cells as an adaptor present in many different protein complexes, or protein-DNA complexes. Our modeling data suggests that GTS1 mediates molecular recognition events mainly through the smaller top surface of domain, which comprises three residues forming a transient complex with other peptides.

### References for Example I

[1] Gibson T J: Cell regulation: determined to signal discrete cooperation. Trends in Biochemical Sciences 2009, 34:471-82.

[2] Hurtley S: Spatial cell biology. Location, location, location. Introduction. Science 2009, 326:1205.

[3] Lambright D G, Sondek J, Bohm A, Skiba N P, Hamm H E, Sigler P B: The 2.0 A crystal structure of a heterotrimeric G protein. Nature 1996, 379:311-319.

[4] Stirnimann C U, Petsalaki E, Russell R B, Müller C W: WD40 proteins propel cellular networks. Trends in Biochemical Sciences 2010, 35:565-574.

[5] Russell R B, Sasieni P D, Sternberg M J: Supersites within superfolds. Binding site similarity in the absence of homology. Journal of Molecular Biology 1998, 282:903-918.

[6] Wilson D K, Cerna D, Chew E: The 1.1-angstrom structure of the spindle checkpoint protein Bub3p reveals functional regions. Journal of Biological Chemistry 2005, 280:13944-13951.

[7] Janda L, Tichý P, Spízek J, Petrícek M: A deduced Thermomonospora curvata protein containing serine/threonine protein kinase and WD-repeat domains. Journal of Bacteriology 1996, 178:1487-1489.

[8] Grigorieva G, Shestakov S: Transformation in the cyanobacterium Synechocystis sp. 6803. FEMS Microbiology Letters 1982, 13: 367-370.

[9] Mishra A K, Puranik S, Prasad M: Structure and regulatory networks of WD40 protein in plants. Journal of Plant Biochemistry and Biotechnology 2012, 21: S32-S39.

[10] Gachomo E W, Jimenez-Lopez J C, Smith S R, Cooksey A B, Oghoghomeh O M, Johnson N, Baba-Moussa L, Kotchoni S O: The cell morphogenesis ANGUSTIFOLIA (AN) gene, a plant homolog of CtBP/BARS, is involved in abiotic and biotic stress response in higher plants. BMC Plant Biology 2013, 13:1-11.

[11] Kotchoni S O, Larrimore K, Mukherjee M, Kempinski C, Barth C: Alterations in the endogenous ascorbic acid content affect flowering time in Arabidopsis thaliana. Plant Physiology 2009, 149: 803-815.

[12] Rice P, Longden I, Bleasby A: EMBOSS: The European molecular biology open software suite. Trends in Genetics 2000 16:276-277.

[13] Larkin M A, Blackshields G, Brown N P, Chenna R, McGettigan P A, McWilliam H, Valentin F, Wallace I M, Wilm A, Lopez R, Thompson J D, Gibson T J, Higgins D G: Clustal W and Clustal X version 2.0. Bioinformatics 2007, 23:2947-2948.

[14] Wernersson R, Pedersen A G: RevTrans: multiple alignment of coding DNA from aligned amino acid sequences. Nucleic Acids Research 2003, 31:3537-3539.

[15] Posada D, Crandall K A: MODELTEST: Testing the model of DNA substitution. Bioinformatics 1998, 14:817-818.

[16] Burnham K P, Anderson D R: Model selection and multimodel inference, a practical information-theoretic approach. Second edition. New York : Springer; 2002.

[17] Guindon S, Gascuel O: A simple, fast, and accurate algorithm to estimate phylogenies by maximum likelihood. Systematic Biology 2003, 52:696-704.

[18] Ronquist F, Huelsenbeck J P: MRBAYES 3: Bayesian phylogenetic inference under mixed models. Bioinformatics 2003, 19:1572-1574.

[19] Nylander J A A: MrAIC.pl. Program distributed by the author. Evolutionary Biology Centre, Uppsala University. 2004a.

[20] Nylander J A A: Bayesian phylogenetics and the evolution of Gall wasps. Comprehensive summaries of Uppsala dissertations from the Faculty of Science and Technology 937. Uppsala University. 2004b.

[21] Wu S, Zhang Y: Recognizing protein substructure similarity using segmental threading. Structure 2010, 18:858-67.

[22] Zhang Y: I-TASSER server for protein 3D structure prediction. BMC Bioinformatics 2008, 9:40.

[23] Kozakov D, Hall D R, Beglov D, Brenke R, Comeau S R, Shen Y, Li K, Zheng J, Vakili P, Paschalidis I C, Vajda S: Achieving reliability and high accuracy in automated protein docking: ClusPro, PIPER, SDU, and stability analysis in CAPRI rounds 13-19. Proteins 2010, 78:3124-3130.

[24] Zimmermann P, Hirsch-Hoffmann M, Hennig L Gruissem W: GENEVESTIGATOR: Arabidopsis microarray database and analysis toolbox; Plant Physiology 2004, 136: 2621-2632.

[25] Baulcombe D C, Saunders G S, Bevan M W, Mayo M A, Harrison B D: Expression of biologically active viral satellite RNA from the nuclear genome of transformed plants. Nature 1986, 321:446-449.

[26] Kotchoni S O, Kuhns C, Ditzer A, Kirch H -H, Bartels D: Over-expression of different aldehyde dehydrogenase genes in Arabidopsis thaliana confers tolerance to abiotic stress and protects plants against lipid peroxidation and oxidative stress. Plant Cell & Environment 2006, 29:1033-1048.

[27] Clough S J, Bent A F: Floral dip: A simplified method for Agrobacterium-mediated transformation of Arabidopsis thaliana. Plant J 1998, 16:735-743.

[28] Ouyang H, Huang X, Lu Z, Yao J: Genomic survey, expression profile and co-expression network analysis of OsWD40 family in rice. BMC Genomics 2012, 13:100.

[29] Xu C, Min J: Structure and function of WD40 domain proteins. Protein Cell 2011, 2:202-214.

[30] Neer E J, Schmidt C J, Nambudripad R, Smith T F: The ancient regulatory-protein family of WD-repeat proteins. Nature 1994, 371:297-300.

[31] Hisbergues M, Gaitatzes C G, Joset F, Bedu S, Smith T F: A noncanonical WD-repeat protein from the cyanobacterium Synechocystis PCC6803: structural and functional study. Protein Sci 2001, 10:293-300.

[32] Valeyev N V, Downing A K, Sondek J, Deane C: Electrostatic and functional analysis of the seven-bladed WD beta-propellers. Evol Bioinform Online 2008, 4:203-16.

[33] Saeki M, Irie Y, Ni L, Yoshida M, Itsuki Y, Kamisaki Y: Monad, a WD40 repeat protein, promotes apoptosis induced by TNF-alpha. Biochem Biophys Res Commun 2006, 342:568-72.

[34] Thornton C, Tang K C, Phamluong K, Luong K, Vagts A, Nikanjam D, Yaka R, Ron D: Spatial and temporal regulation of RACK1 function and N-methyl-D-aspartate receptor activity through WD40 motif-mediated dimerization. J Biol Chem 2004, 279:31357-64.

[35] Garcia-Higuera I, Gaitatzes C, Smith T F, Neer E J: Folding a WD repeat propeller. Role of highly conserved aspartic acid residuesin the G protein beta subunit and Sec13. J Biol Chem 1998, 273:9041-9.

[36] Lau O S, Deng X W: The photomorphogenic repressors COP1 and DET1: 20 years later. Trends Plant Sci 2012, 17:584-93.

[37] Varaud E, Brioudes F, Szecsi J, Leroux J, Brown S, Perrot-Rechenmann C, Bendahmane M: AUXIN RESPONSE FACTOR8 regulates Arabidopsis petal growth by interacting with the bHLH transcription factor BIGPETALp. Plant Cell 2011, 23:973-83.

[38] Meng X, Muszynski M G, Danilevskaya O N: The FT-like ZCN8 Gene Functions as a Floral Activator and Is Involved in Photoperiod Sensitivity in Maize. Plant Cell 2011, 23:942-60.

[39] Li H, He Z, Lu G, Lee S C, Alonso J, Ecker J R, Luan S: A WD40 domain cyclophilin interacts with histone H3 and functions in gene repression and organogenesis in Arabidopsis. Plant Cell 2007, 19:2403-16.

[40] Jain M, Nijhawan A, Arora R, Agarwal P, Ray S, Sharma P, Kapoor S, Tyagi A K, Khurana JP: F-box proteins in rice. Genome-wide analysis, classification, temporal and spatial gene expression during panicle and seed development, and regulation by light and abiotic stress. Plant Physiol 2007, 143:1467-83.

[41] Yu H, Braun P, Yildirim M A, Lemmens I, Venkatesan K, Sahalie J, Hirozane-Kishikawa T, Gebreab F, Li N, Simonis N, Hao T, Rual J F, Dricot A, Vazquez A, Murray R R, Simon C, Tardivo L, Tam S, Svrzikapa N, Fan C, de Smet A S, Motyl A, Hudson M E, Park J, Xin X, Cusick M E, Moore T, Boone C, Snyder M, Roth F P, Barabasi A L, Tavernier J, Hill D E, Vidal M: High-quality binary protein interaction map of the yeast interactome network. Science 2008, 322:104-10.

[42] Collins S R, Kemmeren P, Zhao X C, Greenblatt J F, Spencer F, Holstege F C, Weissman J S, Krogan N J: Toward a comprehensive atlas of the physical interactome of Saccharomyces cerevisiae. Mol Cell Proteomics 2007, 6:439-50.

[43] Miele A E, Watson P J, Evans P R, Traub L M, Owen D J: Two distinct interaction motifs in amphiphysin bind two independent sites on the clathrin terminal domain beta-propeller. Nature Structural & Molecular Biology 2004, 11:242-248.

[44] Miles T D, Jakovljevic J, Horsey E W, Harnpicharnchai P, Tang L, Woolford J L Jr.: Ytm1, Nop7, and Erb1 form a complex necessary for maturation of yeast 66S preribosomes. Mol Cell Biol 2005, 25:10419-32.

[45] Fatica A, Tollervey D: Making ribosomes. Curr Opin Cell Biol 2002, 14:313-8.

[46] Kressler D, Linder P, de La Cruz J: Protein trans-acting factors involved in ribosome biogenesis in Saccharomyces cerevisiae. Mol Cell Biol 1999, 19:7897-912.

[47] Grandi P, Rybin V, Bassler J, Petfalski E, Strauss D, Marzioch M, Schäfer T, Kuster B, Tschochner H, Tollervey D, Gavin A C, Hurt E: 90S pre-ribosomes include the 35S pre-rRNA, the U3 snoRNP, and 40S subunit processing factors but predominantly lack 60S synthesis factors. Mol Cell 2002, 10:105-15.

[49] Fromont-Racine M, Senger B, Saveanu C, Fasiolo F: Ribosome assembly in eukaryotes. Gene 2003, 313:17-42.

[50] Saveanu C, Rousselle J C, Lenormand P, Namane A, Jacquier A, Fromont-Racine M: The p21-activated protein kinase inhibitor Skb15 and its budding yeast homologue are 60S ribosome assembly factors. Mol Cell Biol 2007, 27:2897-909.

[51] Harnpicharnchai P, Jakovljevic J, Horsey E, Miles T, Roman J, Rout M, Meagher D, Imai B, Guo Y, Brame C J, Shabanowitz J, Hunt D F, Woolford J L Jr.: Composition and functional characterization of yeast 66S ribosome assembly intermediates. Mol Cell 2001, 8:505-15.

[51] Hölzel M, Rohrmoser M, Schlee M, Grimm T, Harasim T, Malamoussi A, Gruber-Eber A, Kremmer E, Hiddemann W, Bornkarnm G W, Eick D: Mammalian WDR12 is a novel member of the Pes1-Bop1 complex and is required for ribosome biogenesis and cell proliferation. J Cell Biol 2005, 170:367-78.

[52] Galani K, Nissan T A, Petfalski E, Tollervey D, Hurt E: Real, a dynein-related nuclear AAA-ATPase, is involved in late rRNA processing and nuclear export of 60 S subunits. J Biol Chem 2004, 279:55411-8.

[53] Iouk T L, Aitchison J D, Maguire S, Wozniak R W: Rrb1p, a yeast nuclear WD-repeat protein involved in the regulation of ribosome biosynthesis. Mol Cell Biol 2001, 21:1260-71.

[54] Hofer A, Bussiere C, Johnson A W: Mutational analysis of the ribosomal protein Rpl10 from yeast. J Biol Chem 2007, 282:32630-9.

[55] Ceci M, Gaviraghi C, Gorrini C, Sala L A, Offenhauser N, Marchisio P C, Biffo S: Release of eIF6 (p27BBP) from the 60S subunit allows 80S ribosome assembly. Nature 2003, 426:579-84.

[56] Nilsson J, Sengupta J, Frank J, Nissen P: Regulation of eukaryotic translation by the RACK1 protein: a platform for signalling molecules on the ribosome. EMBO Rep 2004, 5:1137-41.

[57] Ullah H, Scappini E L, Moon A F, Williams L V, Armstrong D L, Pedersen L C: Structure of a signal transduction regulator, RACK1, from Arabidopsis thaliana. Protein Sci 2008, 17:1771-80.

[58] Baum S, Bittins M, Frey S, Seedorf M: Asc1p, a WD40-domain containing adaptor protein, is required for the interaction of the RNA-binding protein Scp160p with polysomes. Biochem J 2004, 380:823-30.

### Example 2

### Functional Characterization of a gst1 Mutant

As discussed at length above in Example 1, GIGANTUS1 (GTS1), a member of Transducin/WD40 protein superfamily, controls seed germination, growth and biomass accumulation through ribosome-biogenesis protein interactions in Arabidopsis thaliana.

### Materials and Methods for Example 2

### Plant Strain, Growth Conditions and Mutant Characterization

Appropriate seeds were surface sterilized and sown on Murashige and Skoog (1×MS) agar plates or on soil and allowed to grow under continuous illumination (120-150 μEm⁻²s⁻¹) at 24° C. . Arabidopsis thaliana (ecotype Col-0) and gts1 knockout mutant (T-DNA SALK_010647) used in this work was grown as described in our previous report (Gachomo et al 2014, BMC Plant Biology 14(1): 37).

Quantitative Real-Time Reverse Transcription-PCR (qRT-PCR)

For the expression level of GTS1, the WT and gts1 mutant cDNAs were used for real-time qRT-PCR performed on Eco real-time PCR system (Illumina, San Diego, Calif., USA) using PerfeCTa SYBR green FastMix (Quanta BioScience, Gaithersburg, Md., USA). Relative GTS1 expression level was assessed in both WT and gts1 mutant using GTS1 specific primers F2 and R2. See Table I.

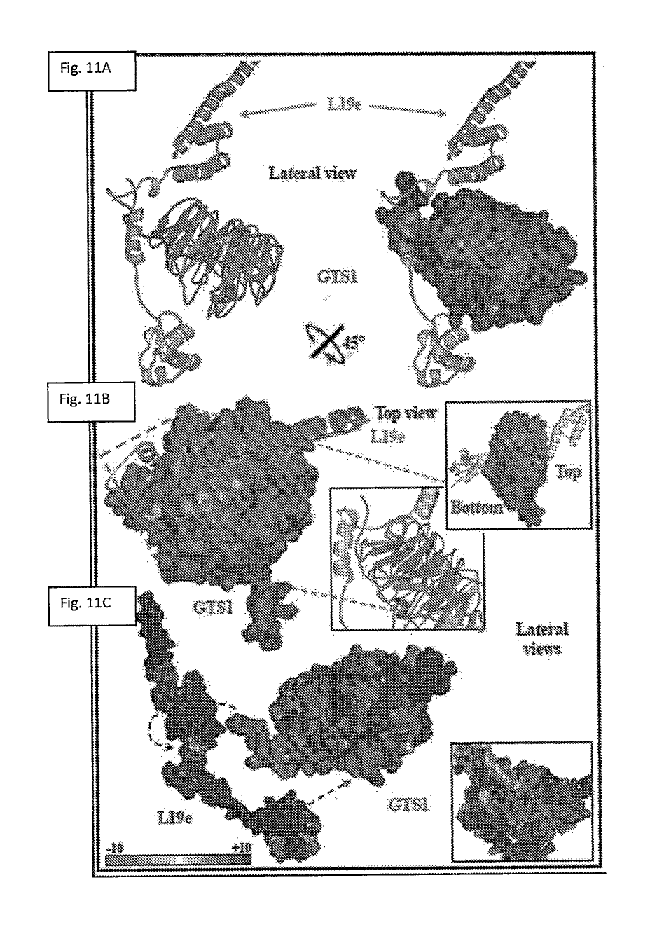

GTS1 is highly expressed during embryo development (Example 1) and negatively regulates seed germination, biomass, growth improvement in plants (FIG. 1) and seed yield in plants (FIG. 12).

Cell Morphogenesis of Growth Pattern of Wild Type vs. Mutant Plant

Confocal image analysis was performed on one week after germination of plate grown plants. Pavement-cell shape analysis was performed by staining the samples with 10 μM of the lipophilic dye, FM464, for 2 hr in darkness under rocking condition. The leaf samples were acquired using confocal microscopy (inverted Leica SP8 confocal microscope at 488 nm, 50% laser power and emission at 600 nm). The F-actin localization was done according to Kotchoni et al. [2]. Images were collected using an inverted Leica SP8 confocal microscope with water-immersion objective. The images were processed and analyzed using ImageJ software as detailed below.

In order to characterize the impact of our gene mutation on plant growth, we developed a possible way of evaluating the consequences of a specific genetic mutation by examining the cellular morphology resulting from the gene knockout, specifically, comparing WT vs. mutant cell shapes. Since a cell fills space within a leaf we theorized that the fractal dimension of the cell boundary as imaged by the dye FM 464 for staining of cell membranes/wall might serve as a suitable discriminant. Cells of the wild type and gts1 mutants were treated with FM 464 and images are obtained as a set of confocal images (a stack). ImageJ was used to isolate and determine the fractal dimension of individual cells using the following procedure:

### Phase 1:

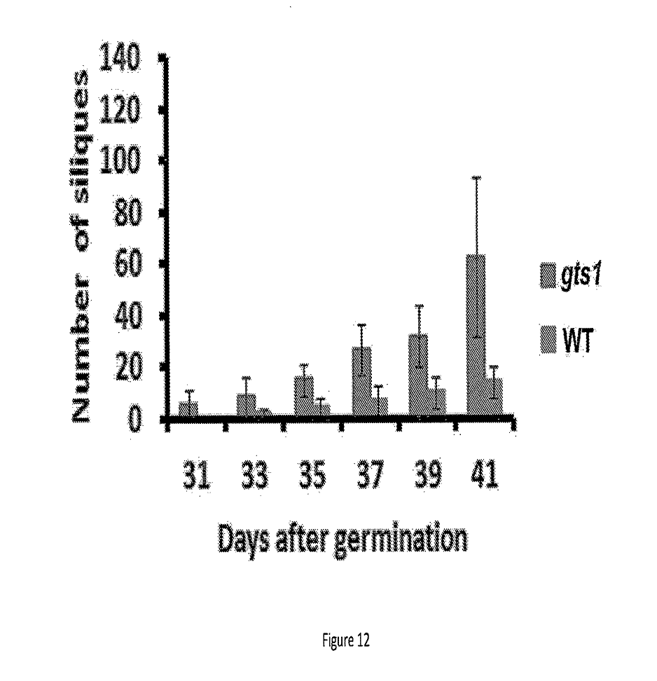

1. A stack was opened and collapsed into a single image based on the ‘Maximum Intensity’ setting to generate a single image of a field and saved as the base image (FIG. 13A, yellow highlight).
2. Cells with complete boundaries within that image were identified and numbered (FIG. 13B). This image was duplicated to provide each cell its own image.
3. Using the tools of ImageJ, each complete cell was identified in the image (FIG. 13C) and the image around the cell was erased (FIG. 13C). With FIG. 13A as the standard, the fluorescent boundary of the isolated cell (FIG. 13C) was thresholded to provide a single cell boundary.
3a) At this step we introduced superimposition model approach because the cell boundary does not uniformly provide a boundary with a singular pixel value to be thresholded. By overlapping the image of the thresholded cell boundary (FIG. 13C) with the original field (FIG. 13A), defects in the discrimination of the cell wall can be identified and fixed. All extraneous pixels and non-essential dead-end projections are removed to generate the single cell (FIG. 13D).
3b) The discriminated cell at this stage is the image of the cell membrane of the cell in question and the adjacent cells (Which cannot be seeing in FIG. 13D). The cell membrane/boundary is measured in pixels.
Erase the outer half, and then check that the cell image is accurate with respect to FIG. 13A by overlapping the 2 FIGS. 13A and 13D (FIG. 13E).
4. At this point, the entire image is made into a binary image. Any existing defects within the resulting boundary are closed with the ImageJ set <Process>Binary>Close>.
5. Obtain a single pixel wide representation of the cell boundary by applying a Canny Edge Detector (FIG. 13F) (which produces an inner and outer edge. Erase the outer edge.
7. Using the step <Process>Binary>Dilate> and suitable values for dilation, the single pixel boundary is now a boundary equal to the width of the cell (FIG. 13G). This image is saved with a unique name and is superimposed onto FIG. 13A (FIG. 13H).

### Phase 2:

8. Within the ImageJ environment, the plug-in Frac-Lac (by A. Karperien) is opened (see the world wide web at rsb.info.nih.gov/ij/plugins/fraclac/fraclac.html).

### STEP 2: FRACLAC

### 1. Set up FracLac for <Standard Box Count>.

1a. Set options for calculating grid calibers.
1b. Select Graphics to Generate (include Lacunarity and Regression Lines).
2. Analyze all images for average fractal dimension
3. Calibrate image to measure perimeter and area of cell in microns or square microns.
4. Plot area vs fractal dimension; and perimeter vs fractal dimension.

In this analysis, the wild type (red, N=46 cells) was compared to gts1 mutant (green, N=55 cells) (See below FIG. 11).

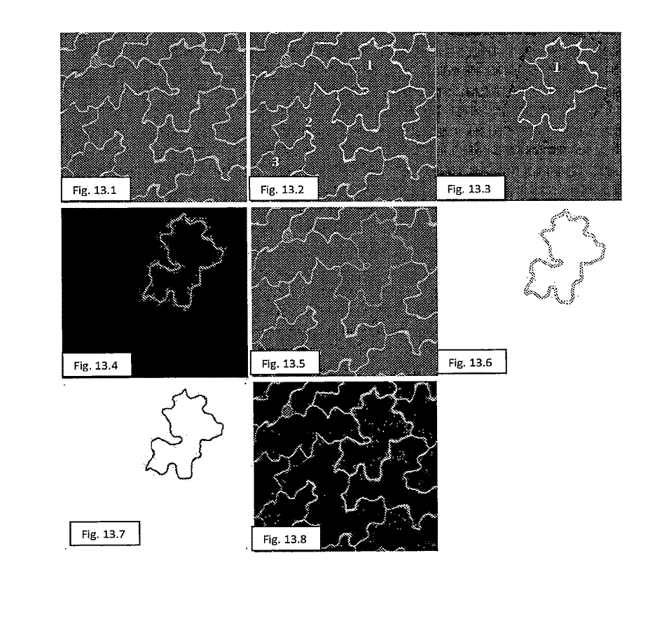

FIG. 14 illustrates the relationship between the average fractal dimension and cell perimeter (left) and average fractal dimension and cell area (right). In these samples, the statistical difference between the two sets of fractal dimensions were significant (P =0.004). 2^{nd} order regression lines are also plotted in the scatter plots.

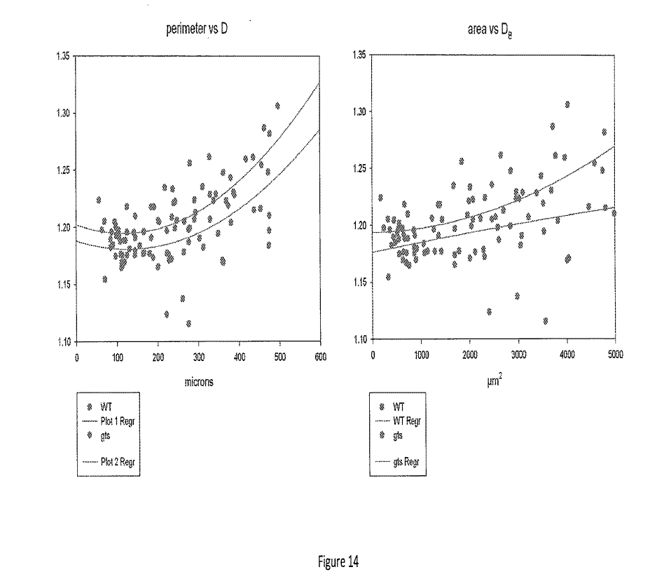

FIG. 15 is a histogram of the area (in 500 μm² bins) vs the fractal dimension. FIG. 15 (along with FIG. 14, right panel) is revealing because it suggests that gts1 mutant has, on the average, a lower fractal dimension as a function of area than the wild type, suggesting the gts1 cells are smaller and simpler in shape than WT cells.

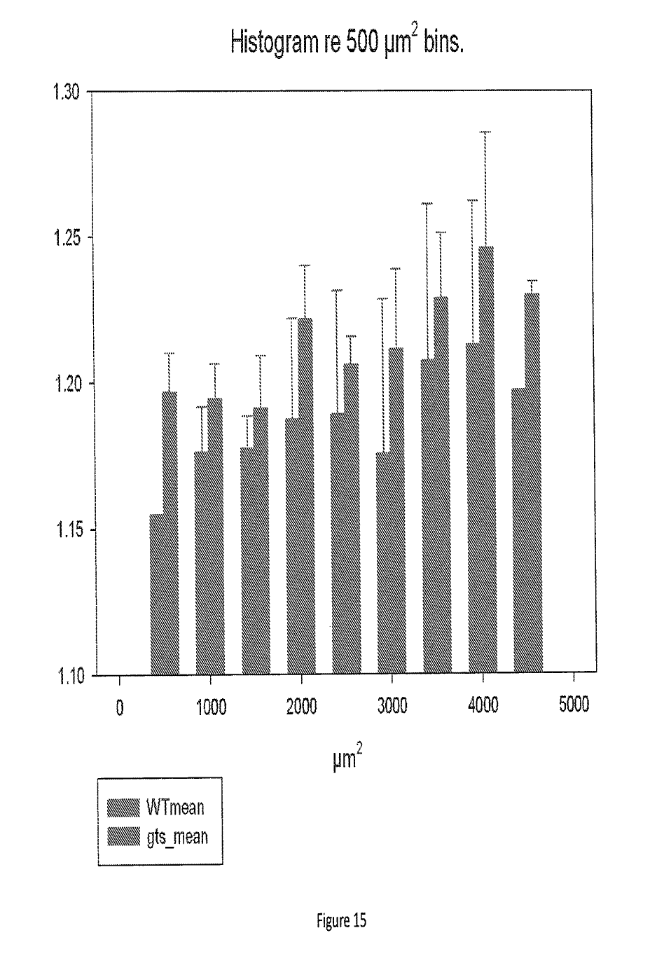

If circularity is considered, then as shown in graph 3, the less circular the cell, the higher the fractal dimension; and for the same value of circularity, WT has a higher fractal than gts1 mutant. This graph (with 1^{st} order regression lines) suggests two important principles: 1) the wild type possesses a higher fractal dimension for same circularity than gts1 mutant; 2) the smaller cells with the lower fractal dimension are more circular than the larger cells. Overall the data suggests that WT cell circularity is more complex than gts1 mutant. The WT cells are overall bigger in size than gts1 mutant (FIG. 16).

In conclusion, the data support the notion that gts1 mutant has a higher rate of endo-reduplication leading to a higher number of cells than WT at the same growth stage, justifying the reason why the overall gts1 mutant plant is bigger than WT at any given time during its growth period compared to WT.

### Preparation of Total Protein Extraction

We next characterized for the first time promising proteins that are affected by this gene knockout resulting in the improved growth, biomass and yield in gts1 plants. Proteins from control and PGPR treated plants were prepared according to Kotchoni et al (2009), with modifications. Leaf materials (2 g) of two week-old plants were harvested and quickly homogenized in ice-cold homogenization buffer [20 mM Hepes/KCl pH 7.2; 50 mM KOAc, 2 mM Mg(OAc)₂; 250 mM Sorbitol; 1 mM EDTA; 1 mM EGTA; 1mM DTT; 1% (v/v) protease inhibitor cocktail (Sigma, St.Louis, Mo.); Kotchoni et al 2009] using a polytron (Brinkman Scientifc, Omaha, Nebr.). The homogenate was filtered through two layers of miracloth and the flow through was spun at 12,000 rpmfor 10 minutes at 4° C. The resulting supernatant was defined as total protein. An aliquot of the total protein was taken to estimate protein concentration using the RC DC protein assay (Bio-Rad) with bovine serum albumin as the standard. The total protein extract was then precipitated overnight in 20% TCA at 4° C. The TCA-precipitated protein was recovered after centrifugation (12,000 rpmfor 10 minutes at 4° C.), air-dried for 10 min and stored at −80° C. until further analysis.

### Protein Sample Preparation—Digestion for LC-MS/MS:

The complex protein sample is enzymatically digested into peptides that are separated by high pressure liquid chromatography (HPLC) and introduced into a mass spectrometer for fragmentation and sequencing to identify the parent proteins. Specifically, sequence grade Trypsin (Sigma) was used to enzymatically digest the samples. The samples were reduced and alkylated. All trypsin digestions were carried out in the Barocycler NEP2320 (PBI) at 50° C. under 20 kpsi for 60 min. Digestions are quenched with 10% TFA. The samples were then spun over C18 columns (MicroSpin, Nest Group), dried, and then resuspended in 97% ddH2O/3% ACN/0.1% FA. 1 μL of volume was used for LC-MS/MS analysis.

### LC-MS/MS Analysis

Tryptic peptides were separated on a nanoLC system (1100 Series LC , Agilent Technologies, Santa Clara, Calif.). The peptides were loaded on the Agilent 300SB-C18 enrichment column for concentration and the enrichment column was switched into the nano-flow path after 5 min. Peptides were separated with the C18 reversed phase ZORBAX 300SB-C18 analytical column (0.75 μm×150 mm, 3.5 um) from Agilent. Column was connected to the emission tip from New Objective and coupled to the nano-electrospray ionization (ESI) source of the high resolution hybrid ion trap mass spectrometer LTQ-Orbitrap XL (Thermo Scientific). The peptides were eluted from the column using the acetonitrile/0.1% formic acid (mobile phase B) linear gradient. For the first 5 min, the column was equilibrated with 95% H₂O/0.1% formic acid (mobile phase A) followed by the linear gradient of 5% B to 40% B in 65 min. at 0.3 ul/min, then from 40% B to 100% B in additional 10 minutes. Column was washed with 100% of ACN/0.1%FA and equilibrated with 95% of H₂O/0.1%FA before next sample was injected. A blank injection was run between samples to avoid carryover.

The LTQ-Orbitrap mass spectrometer was operated in the data-dependent positive acquisition mode in which each full MS scan (30.000 resolving power) was followed by eight MS/MS scans where the eight most abundant molecular ions were selected and fragmented by collision induced dissociation (CID) using a normalized collision energy of 35%. Database search analyses were done using Spectrum Mill v.B.04.00 (Agilent) against NCBI database, species Arabidopsis thaliana.

### Omics Discovery Pipeline (ODP) Workflow

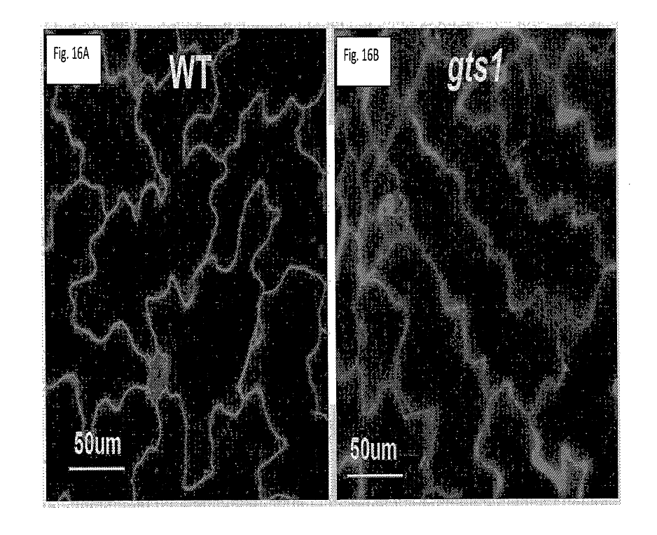

The Omics Discovery Pipeline (ODP) [1] bioinformatics toolset, developed at the Bindley Bioscience Center at Purdue University, was used for LC-MS data analysis. Raw data was converted to the mzXML file format (FIG. 17A, FIG. 17B) and uploaded into the ODP. Each sample was processed by the XMass [2] software application for deconvolution, using a strict set of parameters for peak picking (FIG. 17A, FIG. 17B). The parameters for picking the peaks are as followed: LC Peak Width: 8 (required a peak to be seen in 8 adjacent scans to be picked); m/z Accuracy: 0.05 (Window of m/z variation to be used deisotoping peaks and identifying the same peak across multiple scans); Min/Max Retention Time: 5.0/85.0 (Window of data to be analyzed); and data outside the 5-85 minute range was not processed. Peak lists for each sample were then aligned using the XAlign [3] application for each desired comparison. Aligned peaks were arranged into wild type and mutant groups and passed through a quality control step which eliminated peaks that were not present in all samples for at least one group of replicates. Sets of aligned peaks that did not meet these criteria were dropped, while the rest were normalized using the constant median method. The student's t-test was used for statistical evaluation of peptide peak expression levels between each group. Heatmaps (FIG. 17C) were also generated to show differentially expressed peptides between samples.

Differentially expressed peptides were then cross referenced with identification results from LC-MS/MS analysis using the Agilent Spectrum Mill software package (FIG. 17C). Each sample was searched against the NCBI non-redundant protein database. Peptides with a Spectrum Mill score of 5 or higher and Scored Peak Intensity Value of 70% of higher were considered positives [4]. The m/z and retention time values from the differentially expressed peptides from the ODP/LC-MS analysis were then compared against the parent m/z and retention time values from positive Spectrum Mill hits, providing identification information for differentially expressed peptides.

Generation and Maintenance of New gts1 Maize Germplasm from Tissue Cultures

We applied the knowledge gained from the engineered gts1 mutant plants described in Examples 1 and 2 to generate new gts1 maize germplasm for agricultural and bioenergy (plant-based biofuel production) sustainability.

We generated new maize genotype using a callus production (Kotchoni et al. 2012) procedure developed in our lab, transfection of the callus and regeneration of transgenic plants as detailed below.

For callus induction, seeds were placed on seed germination media (see above) and grown in the dark (the light grown condition was not satisfactory for B73 maize callus induction) at 28-30° C. for 3-5 days, while checking daily for contamination. Seeds were transferred as needed onto fresh media. In these conditions, seeds were expected to germinate in 3-5 days. Alternatively, quickly germinated seeds (within 2-3 days) from the water submersion approach (as described above) were successfully used to induce callus production as well.

Using aseptic techniques, coleoptiles and all roots were removed from germinated seed as close to the cotyledon as possible. This sectioning gives roughly between 0.5-1 cm of residual tissue after removal of un-wanted tissues. The seedling was then cut longitudinally in half through the middle of the meristematic tissues. Both halves of the seedling were then removed from the endosperm and placed on the freshly prepared callus induction media (modified CM4C media: see Table 3) with the exposed/cut surface down (touching the agar). The agar plate was then incubated in the dark (28° C.-30° C.) until the callus was formed. Under this condition, the callus was roughly developed within 5-7 days. The callus was then broken away from non-callus tissues and transferred into fresh callus inducible media.

TABLE 3
Media composition derived from “CM4C-callus inducible media”
fsuitable or callus induction from B73 maize genotype.

| | CM4C | | Modified CM4C | |
|---|---|---|---|---|
| Components | (amount/L) | | (amount/L) | |
| MS salts | 1× | | 1× | |
| MS vitamins | 1× | | 1× | |
| Caseine hydrolysate | 0.1 | g | 0.1 | g |
| Maltose | 40 | g | 40 | g |
| MES | 1.95 | g | 1.95 | g |
| Magnesium chloride | 0.75 | g | 0.75 | g |
| Glutamine | 0.5 | g | 0.5 | g |
| Ascorbic acid | 0.1 | g | 0.1 | g |
| 2,4-D | 0.5 | mg | 0.5 | mg |

| | | | |
|---|---|---|---|
| Picloram | 2.2 | mg | — |

| | | | |
|---|---|---|---|
| NAA | — | 2.2 | mg |

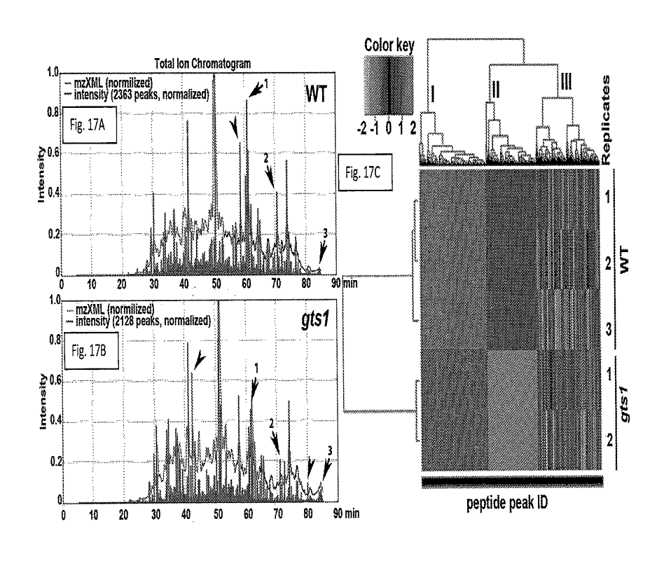

We found that 5 days after germination (DAG) seedlings under dark grown conditions were the best source of materials to induce optimum callus production from B73 maize genotype. From the 5 DAG seedlings we obtained satisfactory callus production from immature B73 maize plant meristematic tissues (FIG. 18), which was used as material for GTS1 gene knockout via RNAi knockout gene approach as described below.

### Construction of Maize GTS1-RNAi Vector and Transformation of Maize Callus

Zm-GTS1 gene was knocked down via RNAi by constructing and inserting the Zm-GTS1-RNAi construct into genomic DNA of B73 maize callus via Agrobacterium tumefaciens mediated transformation to generate a panel of Zm-gts1 callus germplasm.

For the Zm-GTS1-RNAi vector construct, all PCR reactions were performed using a 1 step RT-PCR kit (Thermo Scientific); and all ligations were performed using T4 DNA ligase (New England Biolabs). The Zm-GTS1-RNAi vector construct, by using the conserved fragment of Zm-GTS1 cDNA as depicted in red box of FIG. 3 and stably integrated into B73 maize callus using the transfection method developed in our lab (see detail below) to knockdown GTS1 gene in maize. Since this section of Zm-GTS1 cDNA (FIG. 3, red box) is conserved between maize, rice and Arabidopsis (FIG. 3) the same Zm-GTS1-RNAi vector construct can successfully be used to knockout GTS1 gene in rice as well without any further manipulation. At this level, our genetic manipulation can be employed with the same experimental procedures as detailed above to generate rice gts1 germplasm that can produce more seeds than WT rice under similar growth conditions.

### Transformation of Maize Callus

Double-stranded gene specific RNAi vector constructs were generated by amplifying GTS1 gene fragment from B73 maize cDNA using a gene specific forward primer (flanked with SmaI restriction enzyme site and a T7 RNA polymerase promoter sequences at 5′ end) and a gene specific reverse primer (flanked with the same restriction enzyme site and a T7

RNA polymerase promoter sequences at 3′ end). This PCR product will be inserted between the CaMV 35S promoter and the nopaline synthase (NOS) terminator into the Smal site of the pROK2 vector (Baulcombe et al. 1986) as detailed in our previous work (Kotchoni et al 2006). The 35S promoter of the plasmid was removed in order to express the cDNA fragment from both ends driven by the T7 RNA polymerase promoter flanking both ends of the construct. The resulting construct integrated into B73 maize callus genome using Agrobacterium tumefaciens GV3103 mediated transformation. The transformants were selected on 50 μg mL⁻¹ kanamycin and transferred into new selected media for three generations before proceeding to the regeneration of transgenic plants from callus.

### Transfection of the Calli

The callus was dipped into the Agrobacterium tumefaciens solution using a modified infiltration method (Clough & Bent 1998). The infiltration was performed in osmotic medium (MS basal salts, 0.6 M D-mannitol, 30 g L⁻¹ sucrose, 2 mg L⁻¹ 2, 4-D, and 3 g L⁻¹ phytagel) (Vain et al, 1993) under vacuum condition for 10 min and the suspension further incubated in the dark for 2 days. The transfected calli were then transferred into selected solid media (Table 3) containing kanamycin and allowed to grow further for 5 days. New transgenic calli were transferred into new selected media/plates and gown in the dark for one week. Selected calli were genetically characterized using quantitative real-time reverse transcription-PCR. For qRT-PCR, total RNA of transformed and WT calli were extracted using Trizol and reverse transcribed using qScript cDNA Supermix (Quanta BioSciences, Gaithersburg, Md., USA). Real-time qPCR was performed on Eco real-time PCR system (Illumina, San Diego, Calif., USA) using PerfeCTa SYBR green FastMix (Quanta BioScience, Gaithersburg, Md., USA) to analyze the relative gene expression levels compared to untransformed calli. For control, non transformed calli and calli transformed with empty vector were used for genetic characterization of the germplasm.

Selection of Stable Transformants and Recovery of Transgenic gts1 Maize Plants (Germplasm)

Actively growing kanamycin resistant calli were selected and transferred either to the pre-regeneration medium made of MS basal medium supplemented with 1 mg L⁻¹ NAA (a-naphthalene acetic acid), 1 mg L⁻¹ BAP, and 5 mg L⁻¹ ABA (abscisic acid) for 2 to 3 weeks and then transferred into regeneration medium (MS basal medium, 30 g L⁻¹ sucrose, 2.5 mg L⁻¹ BAP, and 3 g L⁻¹ phytagel, 2 mg L⁻¹ bialaphos). Regenerated shoots were transferred to rooting medium in a Magenta box containing 50 μg mL⁻¹ kanamycin. After about four weeks, rooted plants were transplanted to the potting soil and grown in greenhouse according to our protocol (Kotchoni et al 2012).

All plantlets derived from a single resistant callus and surviving in the rooting medium selection were considered a putative transgenic plant (new germplasm) and can be stably, biochemically, physiologically and genetically studied.

To knock out the GIGANTUS1 gene in maize, we first identified and then cloned the homologous sequence in the maize plant. As detailed below, Maize GIGANTUS1 gene (AZM5 _31259) is a 1652 base pairs (bp) long (see base pair sequence below) and characteristics of the clone confirmation sequence fragment IDs (1a, 1b, 1c, . . . 4b) below the gene sequence bp. In addition, the web link of the gene from maize genome database can be found on the world wide web at maize.jcvi.org/release5.0/asm5.shtml.

```
GIGANTUS1 SEQUENCE
(SEQ ID NO: 1)
      1 AAGTGCTAAT CCTCGTTTCG TTTTGATCAA CGAACAAACC CTAGACCGTT
    51 AAAACCCTTA TCAATTTCGC TGAAACGGCA AATGGAAGAA GTGTCGTCGG
  101 AGATGGAAGT GGAGGTTCAA AATCGTCAGC TTTCGGATTC TTCTCCGGCT
  151 CAAAACGTGA AGAAATTTGG GCTCAAGAAC TCTATTCAGA CCAACTTCGG
  201 CTCTGACTAC GTCTTCCAGA TTGTCCCAAA GTAAAAGCTT TCTGTTCTCT
  251 CTTTTCGATA ACCATACTAA TTAGTTTACT GAAAGTGAAC CCAACTCTGG
  301 TTTTGTGACA GAATCGATTG GACAGCAATC GCGGTTTCCT TATCAACCAA
  351 CACCGTCAAG CTTTACTCGC CGGTGACTGG TCAGTACTAC GGCGAATGTA
  401 AAGGCCACTC CGATACCGTC AATCAGATTG CGTTTTCCTC CGACTCGGCG
  451 GCGTCTCCTC ACGTTTTGCA CTCTTGTTCC TCCGATGGTA CCATTAGATC
  501 TTGGGACACT CGAAGTTTCC AACAGGTGAG TTTTTTTTTT TTTATATAAA
  551 TTCTGTCGTA AATGTGATTC CATTTTCGGA ACTCATTGAA TTTAAGTTGA
  601 TCATGGGTGT TTCAGGTTTC GCGTATTGAT ACTGGGAATG ATCAGGAGAT
  651 TTTCAGCTTC TCCTATGGAG GTGCTGCAGA TAATCTTCTG GCTGGTGGAT
  701 GCAAAGAGCA GGTTAGGCAT CTCAATCCTG TGGAATCGTG TTTTTTTTTT
  751 TGTTTCTCTA AATGTTTAAA AAGGAGTACA CTTTGAGAAA GAAAAAAGGC
  801 TAGTTTTGAG CATTTTCTAT GTTTCAGCTT GTTACAATCT GAAGCGATGG
  851 TGCAGGTTCT TTTGTGGGAC TGGAGAAACT CAAAGCAAGT TGCTTGTTTA
  901 GAGGAATCCC ACATGGACGA CGTCACTCAG GTTTGTTGCT CTCTCATTCT
  951 TTCTGCTCAA GCGGGAAAGG AACTAGTTTG TTGTTAGTTT ATAGATGTCA
1001 ACTCTGTTCT TAATTATAGG TACACTTTGT TCCTAACAAG CCCAACAAGC
1051 TTCTTTCCGC TTCCGTGGAT GGATTGATAT GTCTTTTTAA CACTGAAGGT
1101 GATATCAATG ACGATGATCA TCTGGAATCT GTAAGTTTTA GATTTAAGGT
1151 ATCAGCCTTC TTGCTGCATC AAGATAGTAG ATGAGAGTTC TGAACCATTT
1201 CTGCCTTGGT CGCTCTTTAG GTCATCAACG TGGGAACTTC AATTGGCAAA
1251 ATAGGTTTCC TCGGAGATGG CTATAAAAAG CTCTGGTGTC TCACTCATAT
1301 AGAAACTCTA AGGTACTCTT TCACGCCCTC CTCTCCAACT GTAGTCTGTT
1351 ACTTGTGCTG TTTTTAATTT CTTTACTCCT TTCTAGTATT TGGAATTGGG
1401 AAGACGGAAG CTGTGAAGTC AACCTAGAGA AAGCTCGGGA ACTCGCATCA
1451 GATAGTTGGA CTCAAGATAA TGTAACAATC TCATTCTTTC CCTTCTAGTA
1501 AAACATTAAC ACTAACCAGA AACCTAATGG TTACTTACAG AGTTGAACTT
1551 TCCCATTACT CTTTTGCAGG TTGATTATTT TGTTGATTGT CATTGTCCAG
1601 GAGGTGAAGA TTTATGGGTG ATCGGTGGAA CGTGCGCAGG AACCGTTGGT
1651 TATTTTCCGG TGAACTACAA ACAGCCTGGA TCAATCGGTA CCGCTGAAGC
1701 TATTCTTGGT GGAGGTCACA TAGATGTAGT AAGGAGCGTT CTGCAGATGC
1751 CAGGCGAGTA TGGAGGAGCT GCAGGGTTAT TTGGATGGAC TGGGGGTGAA
1801 GATGGGCGAC TATGTTGCTG GAAGTCGGAT GAGGATGCTA CCGAGATTAA
1851 CCGGTCTTGG ACTTCAAGTG AGTTGGTTGT TAAGCCGCCA AGGAACCGCA
1901 AGAAAAACAG ACACTCTCCT TACTAGTAAA CAGATACTAC TTTTTGGGTC
1951 TTGTGTCTTT TATAAAAGTT GAGACTGAAG AGGACAGTTT CCTTTATGTC
2001 TCTTGTTCTT GTTATTATTA AAAGGCAAAG TTGTTTAAAT GAAATATTGA
2051 AGAAAGATTA CATC
Cloning of maize GIGANTUS1 gene
(SEQ ID NO: 2)
AZM Report: AZM5_31259 (Maize GTS1)
>AZM5_31259 (Maize GTS1)
TGAAATTTCTTGTGACACGCCTCCTCTTAGCAAAGATATCTGGCAACATTTAGAGACAGT
GAACAGTTGGATTTAGAACATAGAGAACTTCAGACAAACAGGTGTACTTACTGGTACAAA
AAAGAGAAACAAAAAAGCTTGGCATACAAGCACACTAATCATTAATGGCATATCAAATGC
TGAAATGCATGCTAAGTGGTAAAACTCTTGACAAGACACATGTAGCATATAATGAAGTGC
TATATCATTACATTAGTAGCTACTTAGATAAAAAATATAAAACCTATTCAACATACAATA
ATATTTTTCACATTGGAATGAAGAATTCATACTGTCATAAAGCTGTAAGAAAACTCTGTT
AGCTAAGTTCAGCAAACTGCATAAGTTCATTGTAATGTTGGTCACAAAGGCAATCACAAA
AATGACATCACCTTATTGAGTTGAACTTCCATTTACTTATCTTTTTTTTCTTTYTTTTGG
CGAAAAGGCATCTAGCCGAGTTGGTTAGACGGTCTGAGTCCTCAAGTCCCGACTTCGACT
ACCGTGGAAGCGGATTTCAGGCTGTGGTTAAAAAAACCTTGTCTATCCCACGCCAAAGCA
CGAGTCTAAGGCCCGGCCCCAGTCGCGGTCATTCTCACATGGGCTACGGTGCCGTTGTGT
ATGGGTGGGACAGAGGTTCAGGAGTTTCTCGACCTGTGTGAGAAGGTCTTCTTTTTAATA
CAATGCCCAGGGGCTGTCTTACCTCCCGTAGGTCGAGTTTTTCTTTTGAACCGGAAGAAA
GTTTTGAGTTGCCCAGTATAAAGAAAAAGTAGGTACAAACTACGGATAAATATACTCATG
TTCCATCAAATGGGATCAATGGCCAACCCAAATTCAAGCATGAAAATAAAAACAGAACTT
GTTTTAGTAGTAGAATTTACAAAGCGAACAGAAGAATTAGTCATGGCGCTGTAACGATTT
TAAGCAGAAGTAGGGCAGTGTTCCAATATGAATGCATAAGCAGTAGCAATAAAGTGCATA
CGGCAACATATATTAATCTAAACGTAGGACAAATGACTAAATGAAGTGAACAAGAATATA
GTCATTATGTTAACAGGAACATGAAAAAATCGAACATAAGAGAACAAAACAGAAATGTTG
ACTAATTTCCATTAGCATGAACAATATCATAACCATTTTAGGAGCACACAATCATGGGCA
CAATGTTCAGATGTTCTACACCTTTAGCCTGTAGGGTAATATGGATATATGATTATCTTT
TTCATTTTGATTGTGAAATAAGTAGATGTGGTAGTCTTATTAAGCAACCATACACACATA
CAAGGAAAATTGAAATGCTGACAAGAATTTTCAATAAACATAAAGTATTAGAAAACAACC
TGCTTAAAGTTTCTTGTATCCCAAGCTCTGACAGTTCCATCAGAAGAACAAGAGCAGATT
ACTTGTGGTGACGATGGAGCTGAGAAGGAGATTTCATGGATTGTTCCTTCGTGTCCTTTA
CATTCCCCTAGGTACTGTCCGGTTTCTGGAGAATAAAACTTTAGTGCATTTGTTGACAAG
GAAACTGCAAGCGTTGAGATCTCCTGGCTTGTTACCAGATGTATCCAAGGAGAAGCAAAA
GAGCATCAGCATCAGTACTAGAAGCACAGATA
1==================================AZM5_31259===============================1652
1a>
.............................<1b...
2a>
.......................................<2b
3>
4a>
.................<4b
```

| # | Src | Seq Id | GB# | Clone | left | right | |
|---|---|---|---|---|---|---|---|
| 1a | TIGR* | PUDAY44TD | BZ722318 | PUDAY44 | 1 | 769 | High- |
| | | | | | | | CoT |
| | | | | | | | library |
| 1b | TIGR | PUDAY44TB | BZ722316 | PUDAY44 | 601 | 703 | High- |
| | | | | | | | CoT |
| | | | | | | | library |
| 2a | TIGR | PUIGR70TD | CG054090 | PUIGR70 | 7 | 609 | High- |
| | | | | | | | CoT |
| | | | | | | | library |
| 2b | TIGR | PUIGR70TB | CG054086 | PUIGR70 | 798 | 1504 | High- |
| | | | | | | | CoT |
| | | | | | | | library |
| 3 | TIGR | PUHDK18TB | CC406311 | PUHDK18 | 214 | 340 | High- |
| | | | | | | | CoT |
| | | | | | | | library |
| 4a | TIGR | PUHQC49TB | CC410276 | PUHQC49 | 400 | 1326 | High- |
| | | | | | | | CoT |
| | | | | | | | library |
| 4b | TIGR | PUHQC49TD | CC410280 | PUHQC49 | 736 | 1652 | High- |
| | | | | | | | CoT |
| | | | | | | | library |

*TIGR The Institute for Genomic Research

In Table 4, we highlight in italics and bold, the high percentage identity and percentage similarity of the maize GIGANTUS1 gene with Arabidopsis GIGANTUSJ (At2g447790) confirming the proper identification and characterization of this gene is maize.

TABLE 4
Best hits for AZM5_31259

| | % | % | Seq | Seq | Hit | Hit | |
|---|---|---|---|---|---|---|---|
| Hit | ID | Sim | Left | Right | Left | Right | Hit Name |
| [symbol] | [symbol] | [symbol] | [symbol] | [symbol] | [symbol] | [symbol] | [symbol] [symbol] [symbol] |
| | | | | | | | [symbol] — [symbol] |
| | | | | | | | [symbol] [symbol] [symbol] |
| UP\|O65367 | 70.49 | 86.89 | 1568 | 1377 | 57 | 122 | RGA-like protein |
| UP\|Q944S2 | 70.49 | 86.89 | 1568 | 1377 | 57 | 122 | At2g47790/F17A22.18 |
| | | | | | | | (Expressed protein) |
| UP\|Q84XQ8 | 69.09 | 85.45 | 1568 | 1377 | 8 | 67 | RGAL (Fragment) |

### Construction of Vector and Transformation of Maize Callus

For the construction of the GTS1-RNAi vector, we employed the well-established construct vector developed in our lab that worked well for our previous RNAi knockout mutants in green algae (Gachomo et al., 2014).

All PCR reactions were performed using a 1 step RT-PCR kit (Thermo Scientific); and all ligations were performed using T4 DNA ligase (New England Biolabs). For the GTS1-RNAi vector construct, an 582 bp fragment of GTS1 was first amplified from the cDNA of wild type (B73) maize using forward (For) primer (gene specific, flanked with NOT1 and T7 RNA polymerase promoter sequences at 5′ end) and reverse (Rev) primer (gene specific, flanked with NOT1 and T7 RNA polymerase promoter sequences at 3′ end) (FIG. 1, Table 5). The PCR product was ligated into NOT1-digested plasmid MWpBacFPNS. The resulting recombinant plasmid, MWpBacFPNS-GTS1-RNAi, was used to transform wild type (B73) maize callus via electroporation. The transformants were selected and maintained on callus growth media (Table 3) supplemented with 1 μg ml⁻¹ selected antibiotic. Selected callus mutants were genetically characterized using quantitative real-time reverse transcription-PCR (qRT-PCR).

TABLE 5
Sequences of oligonucleotide primers used for
generating the maize GTS1 genotypes

| Primer name | Sequence | # of bases | Used for |
|---|---|---|---|
| For. | TCCATCGTCACCACAAGTAATC *(10) | 22 | GTS1 cDNA specific |
| Rev. | CGGTCATCAGGCACAGAATAG (11) | 21 | GTS1 cDNA specific |
| For. | GCGGCCGCTAATACGACTCACTATAGGTCCATCGTC | 47 | PCR, RNAi |
| | ACCACAAGTAATC (12) | | |
| Rev. | GCGGCCGCTAATACGACTCACTATAGGCGGTCATCA | 48 | PCR, RNAi |
| | GGCACAGAATAG (13) | | |
| Actin-F | GAGGATGATGATGCAGCGATAG (14) | 22 | qRT-PCR |
| Actin-R | TTTGGCGCGGAATGAAGA (15) | 18 | qRT-PCR |

NOT1 site = in bold; *numbers in parentheses are SEQ ID NOS:
T7 sequence = underlined
GTS1 cDNa specific = color coded (green and red).

Quantitative Real-Time Reverse Transcription-PCR (qRT-PCR)

Total RNA of transformed and WT callus maize was extracted using Trizol and reverse transcribed using qScript cDNA Supermix (Quanta BioSciences, Gaithersburg, Md., USA). Real-time qPCR was performed on Eco real-time PCR system (Illumina, San Diego, Calif., USA) using PerfeCTa SYBR green FastMix (Quanta BioScience, Gaithersburg, Md., USA). Mutants were screened for relative GTS1 expression level compared to untransformed maize (B73) callus using the GTS1 specific primers For and Rev (Table 5).

### Generation and Characterization of Genomic Insertion of GTS1-RNAi in Wild Type (B73) Maize Callus Background

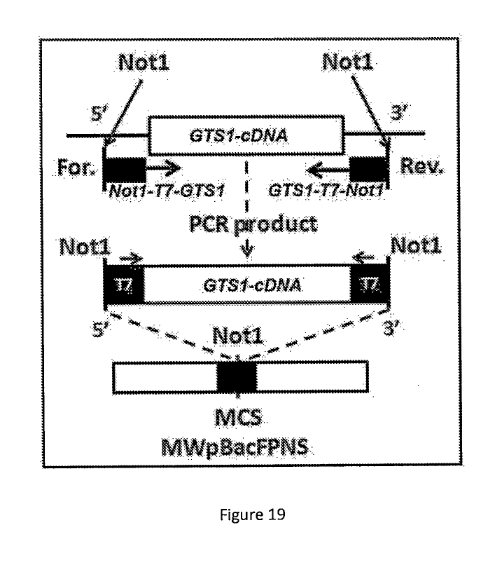

After transformation of maize callus, the insertion of the GTS1-RRNAi construct into the genomic DNA was assessed using the RNAi primers (Table 5). Genomic DNA was extracted from wild type (B73) callus and GTS1-RNAi transformed callus and used to amplify the insertion. As shown in FIG. 20, we successfully amplified the GTS1-RNAi fragment (lane 2) from transformed callus and, as expected, the wild type (lane 1) shows no band of the insert. The lower bands indicate the actin control fragments from both wild type and transformed callus.

Stable integration of the construct into the genomic DNA was achieved by growing the transformed callus for several generations for three months and checking/confirming every time for the GTS1-RNAi insertion in subsequent progenies.

### GIGANTUS1 Gene Expression Analysis in Transformed Callus Versus Wild Type (B73) Callus.

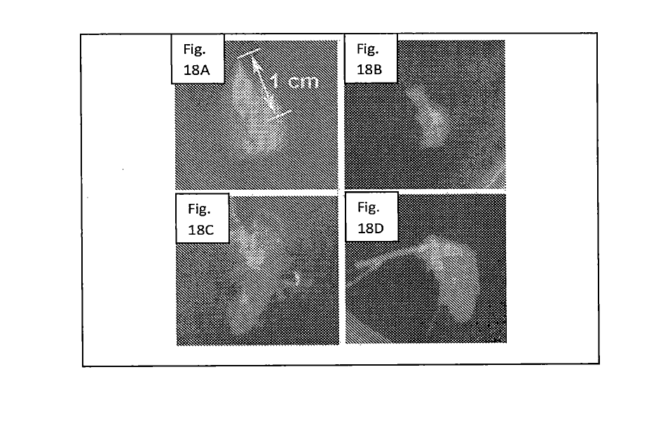

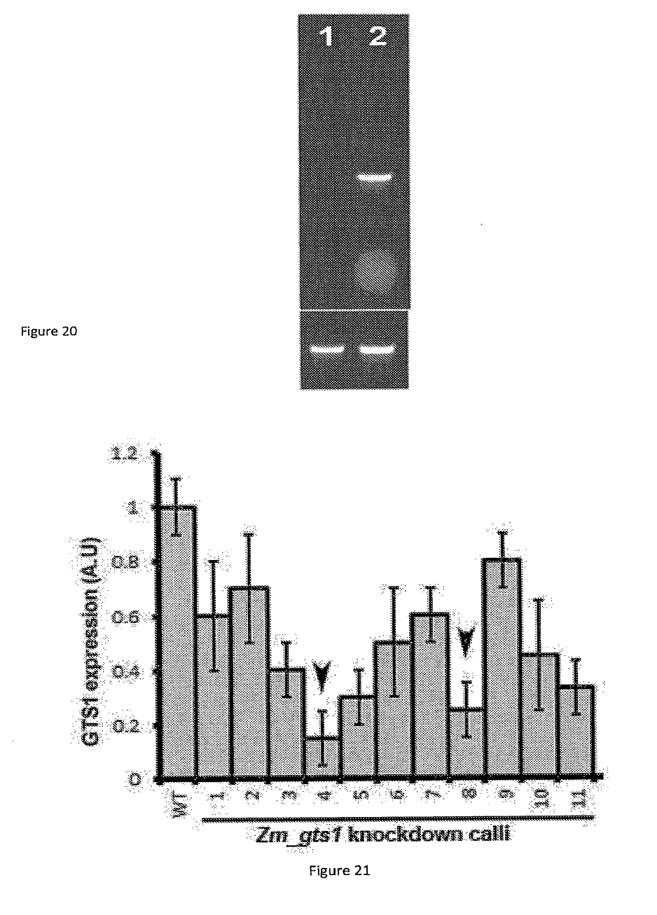

The GIGANTUS1 transcript was knocked down via introduction of the GTS1-RNAi construct into genomic DNA of wild-type callus generating a panel of mutants. These were compared to wild type non-transformed callus (FIG. 19, FIG. 21). From this knockout data we selected two knockout mutants for further analysis based on their high level of GTS1 knockdown expression. The two selected calli, Zm_gts1-4 and Zm_gts1-8 displayed 15% and 25% of WT GTS1 expression, respectively (FIG. 21).

### References for Example 2

[1] Riley C P, Gough E S, He J, Jandhyala S S, Kennedy B, Orcun S, Ouzzani M, Buck C, Roumani A M, Zhang X: The Proteome Discovery Pipeline—A Data Analysis Pipeline for Mass Spectrometry-Based Differential Proteomics Discovery.The Open Proteomics Journal 2010, 3:8-19
[2] Zhang X, Asara J M, Adamec J, Ouzzani M, Elmagarmid A K: Data pre-processing in liquid chromatography-mass spectrometry-based proteomics. Bioinformatics 2005, 21:4054-4059.

[3] Zhang X, Hines W, Adamec J, Asara J M, Naylor S, Regnier F E: An automated method for the analysis of stable isotope labeling data in proteomics. J Am Soc Mass Spectrom 2005, 16:1181-1191.

[4] Kapp E A, Schutz F, Connolly L M, Chakel J A, Meza J E, Miller C A, Fenyo D, Eng J K, Adkins J N, Omenn G S, Simpson R J: An evaluation, comparison, and accurate benchmarking of several publicly available MS/MS search algorithms: sensitivity and specificity analysis. Proteomics 2005, 5:3475-3490.

[5] Kotchoni S. O., Zakharova T., Mallery E. L., El-Din El-Assal S., Le J., and Szymanski D. B. (2009). The association of the Arabidopsis actin-related protein (ARP) 2/3 complex with cell membranes is linked to its assembly status, but not to its activation. Plant Physiology 151(4): 2095-2109.

[6] Kotchoni S. O., Noumavo P. A., Adjanohoun A., Russo D. P., Dell'Angelo J., Gachomo E. W., Baba-Moussa L. (2012). A simple and efficient seed-based approach to induce callus production from B73 maize genotype. American Journal of Molecular Biology 2(4): 380-385.

[7] Kotchoni S O, Kuhns C, Ditzer A, Kirch H -H, Bartels D (2006). Over-expression of different aldehyde dehydrogenase genes in Arabidopsis thaliana confers tolerance to abiotic stress and protects plants against lipid peroxidation and oxidative stress. Plant Cell Environ29:1033-1048.

[8] Baulcombe D C, Saunders G S, Bevan M W, Mayo M A, Harrison B D (1986). Expression of biologically active viral satellite RNA from the nuclear genome of transformed plants. Nature 321:446-449.

[9] Vain, P., M. D. McMullen, and J. J. Finer. 1993. Osmotic treatment enhances particle bombardment-mediated transient and stable transformation of maize. Plant Cell Rep. 12: 84-88.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An isolated nucleic acid molecule comprising a GIGANTUS1 (gts1)-encoding nucleic acid sequence of SEQ ID NO: 1 or a recombinantly produced sequence having at least 70% identity to SEQ ID NO: 1, cloned into an expression vector, said nucleic acid optionally being mutated or disrupted.

2. An isolated nucleic acid molecule comprising a GIGANTUS1 (gts1)-encoding nucleic acid sequence of SEQ ID NO: 2 or a recombinantly produced sequence having at least 70% identity to SEQ ID NO: 2, cloned into an expression vector, said nucleic acid optionally being mutated or disrupted.

3. A cDNA produced by reverse transcription of an mRNA encoded by the nucleic acid sequence of claim 1.

4. A cDNA produced by reverse transcription of an mRNA encoded by the nucleic acid sequence of claim 2.

5. The isolated nucleic acid molecule of claim 1 isolated from a plant selected from Arabidopsis, Maize and Rice.

6. An isolated siRNA molecule directed to the gstl encoding nucleic acid of claim 1.

7. The expression vector of claim 1 wherein said vector is selected from the group of vectors consisting of plasmid, cosmid, baculovirus, bacteria, yeast and viral vectors.

8. A host cell transformed with an expression vector of claim 7.

9. A host cell of claim 8, wherein said host cell is selected from the group consisting of Arabidopsis, rice, maize, wheat, soybean, tomato, potato, barley, canola, bacteria, yeast, insect and mammalian cells.

10. A host cell of claim 9, wherein said host cell is a maize cell or a rice cell.

11. A host cell of claim 9, wherein said host cell is an Arabidopsis thaliana cell.

12. A plant regenerated from the host cell of claim 10, wherein said plant exhibits at least one of increased biomass, increased seed production, early flowering and robust growth development.

13. A method for identifying agents which modulate gts1 activity or expression levels in a host cell, comprising the steps of:
a) introducing a gts1-encoding nucleic acid into said host cell;
b) treating said host cell with agents suspected of modulating gts1 activity and/or expression level; and
c) assaying gts1 activity and/or expression level in the presence and absence of said agent in said host cell or extracts thereof, agents which alter gts1 activity and/or expression relative to untreated controls being identified as gts1 modulating agents.

14. The method of claim 13, wherein said agent disrupts gts1 binding to L19e.

15. The method of claim 13, wherein said agent disrupts gts1 binding to Nop16.

16. A method for modulating one or more features selected from increased biomass, increased seed production, early flowering and robust growth development in a plant comprising disrupting or inhibiting expression of a gts1 encoding nucleic acid molecule of SEQ ID NO: 1 in a plant cell.

17. A method to inhibit gts1 function in a plant, said method comprising the introduction of a mutated gts1-encoding nucleic acid into said plant, said mutated gts1-encoding nucleic acid encoding a non-functional gts1 protein.

18. The method of claim 13, wherein said mutated gts1-encoding nucleic acid encodes an RNAi directed to SEQ ID NO: 1.

19. A plant produced by the method of claim 16.

20. A plant produced by the method of claim 18

21. A method of creating improved maize germplasm by inhibiting expression of gts1 comprising
a) introducing a nucleic acid construct which inhibits expression of the gts1 nucleic acid in a plant cell;
b) generating transgenic maize plant callus from said plant cell; and
c) regenerating a plant from said plant cell, wherein said plant exhibits one or more phenotype selected from the group consisting of early flowering time, biomass accumulation, improved crop yield, enhanced early growth rate and resilience to drought stress.

22. A transgenic maize plant produced by the method of claim 21.
